(19) United States
(12) Patent Application Publication
(10) Pub. No.: US 2026/0199364 A1
(43) Pub. Date: Jul. 16, 2026

(54) USE OF A SYK INHIBITOR FOR THE TREATMENT OF SICKLE CELL DISEASE

(71) Applicant: Rigel Pharmaceuticals, Inc., South San Francisco, CA (US)

(72) Inventors: Vadim MARKOVTSOV, San Mateo, CA (US); Esteban MASUDA, Menlo Park, CA (US)

(21) Appl. No.: 19/161,211

(22) PCT Filed: Mar. 1, 2024
(86) PCT No.: PCT/US2024/018228

§ 371 Date: Aug. 29, 2025

Related U.S. Application Data
(60) Provisional application No. 63/449,306, filed on Mar. 1, 2023.

(51) Int. Cl. A61K 31/5383; A61K 9/00; A61K 31/17; A61P 7/06

(52) U.S. Cl. CPC A61K 31/5383; A61K 9/0053; A61K 31/17; A61P 7/06

(57) ABSTRACT

Provided herein, among other things, is a method for treating sickle cell disease by administering fostamatinib, an active component thereof or a pharmaceutically acceptable salt thereof to a subject.

### CROSS-REFERENCE TO RELATED APPLICATIONS

This application claims the benefit of the earlier filing date of U.S. Provisional Application No. 63/449,306, filed on Mar. 1, 2023, which is incorporated herein by reference in its entirety.

### INTRODUCTION

Inflammation is a principal component of the pathophysiology of sickle cell disease. The spectrum of sickle cell disease inflammation is broad, impacting many pathways in virtually all organ systems. Systemic inflammation in subjects is evident by elevated steady-state concentrations of several inflammatory markers, as well as raised plasma Tumor Necrosis Factor-α (TNF-α), which are elevated further during acute illness. Increased leukocyte count is associated with the development of many severe complications of SCD including acute chest syndrome and stroke, and with a higher mortality rate. Acute inflammation in major organs is common; the most widely described is a condition in the lung known as acute chest syndrome, which shares many similarities with acute respiratory distress syndrome. Acute chest syndrome is the second most common reason for hospital admission and the leading cause of referral to intensive care units.

There is a continuous need for new treatments for sickle cell disease, particularly for treatments for immunological responses that occur in the disease.

### SUMMARY

Disclosed herein is a method for reducing immunological responses resulting from sickle cell disease by administering an inhibitor of Spleen tyrosine kinase (SYK), e.g., fostamatinib, an active component thereof or a pharmaceutically acceptable salt thereof to a subject. Without wishing to be bound to any particular theory, the compound is believed to quell immunological responses associated with the disease, thereby treating those subjects.

### BRIEF DESCRIPTION OF THE FIGURES

FIG. 1 shows embodiments of targets of fostamatinib.

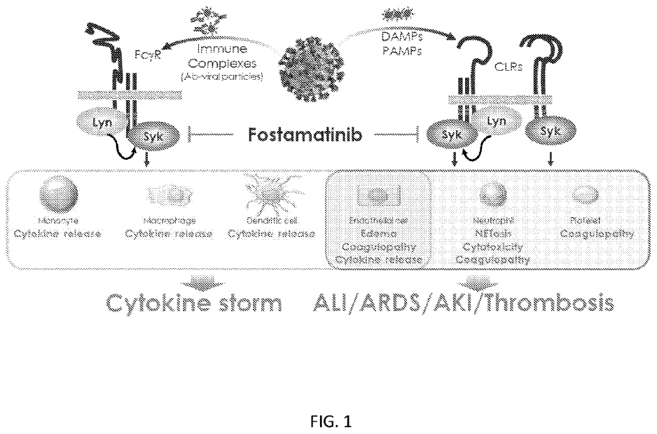

FIG. 2 shows embodiments of targets (*) of fostamatinib including CLEC-dependent cytokine release by innate immune cells, immune complex driven Fc receptor mediated monocyte or endothelial cell activation, and neutrophil activation and NETosis.

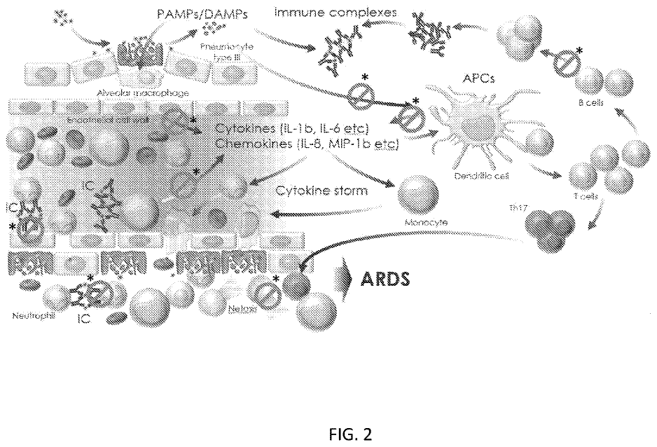

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is specifically contemplated. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

The nomenclature used herein to name the subject compounds is illustrated in the Examples herein. This nomenclature has generally been derived using the commercially-available AutoNom software (MDL, San Leandro, CA.).

### Terms

The following terms have the following meanings unless otherwise indicated. Any undefined terms have their art recognized meanings.

The term “acute respiratory distress syndrome” or “ARDS” refers to a syndrome characterized by a severe shortness of breath, labored and unusually rapid breathing, low blood pressure, confusion and extreme tiredness. This syndrome can be diagnosed based on a PaO2/FiO2 ratio of less than 300 mmHg despite a PEEP of more than 5 cm H₂O (Fan et al JAMA. 319: 698-71).

ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. Severe shortness of breath—the hallmark of ARDS—usually develops within a few hours to a few days after exposure or infection. Many people who develop ARDS do not survive, and the risk of death increases with age and severity of illness. Of the subjects that survive ARDS, some completely recover while others have lasting damage to their lungs. ARDS may be referred to as Acute Lung Injury (ALI) in some publications.

The term “treatment” refers to a reduction in symptoms as well as prophylactic treatments.

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. As used herein, “lower alkyl” means (C1-C8) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. As used herein, “lower alkanyl” means (C1-C8) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. As used herein, “lower alkenyl” means (C2-C8) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. As used herein, “lower alkynyl” means (C2-C8) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In some embodiments, the alkyldiyl group is (C1-C8) alkyldiyl. Specific embodiments include saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In some embodiments, the alkyleno group is (C1-C8) or (C1-C3) alkyleno. Specific embodiments include straight-chain saturated alkano groups, e.g., methano, ethano, propano, butano, and the like.

“Heteroalkyl,” Heteroalkanyl, “Heteroalkenyl,” Heteroalkynyl, “Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or (C1-C8) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or (C1-C8) alkyl.

“Parent Aromatic Ring System” refers to an unsaturated cyclic or polycyclic ring system having a conjugated Tr electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like.

“Aryl” by itself or as part of another substituent refers to a monovalent aromatic hydrocarbon group having the stated number of carbon atoms (i.e., C6-C15 means from 6 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C6-C15) aryl, with (C6-C10) being more typical. Specific exemplary aryls include phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C6-C15) arylaryl is an arylaryl group in which each aromatic ring comprises from 6 to 15 carbons, e.g., biphenyl, triphenyl, binaphthyl, phenylnaphthyl, etc. In some embodiments, each parent aromatic ring system of an arylaryl group is independently a (C6-C15) aromatic, more preferably a (C6-C10) aromatic. Specific exemplary arylaryl groups include those in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. In some embodiments, the aromatic ring systems are (C6-C15) aromatic rings, more typically (C6-C10) aromatic rings. A particular exemplary biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In some embodiments, the arylalkyl group is (C7-C21) arylalkyl, e.g., the alkanyl, alkenyl or alkynyl moiety of the arylalkyl group is (C1-C6) and the aryl moiety is (C6-C15). In some specific embodiments the arylalkyl group is (C7-C13), e.g., the alkanyl, alkenyl or alkynyl moiety of the arylalkyl group is (C1-C3) and the aryl moiety is (C6-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Specifically excluded from the definition of “parent heteroaromatic ring system” are benzene rings fused to cyclic polyalkylene glycols such as cyclic polyethylene glycols. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaiyl is a heteroaryl-heteroaryl group in which each parent heteroaromatic ring system comprises from 5 to 15 atoms, e.g., bipyridyl, tripuridyl, etc. In some embodiments, each parent heteroaromatic ring system is independently a 5-15 membered heteroaromatic, more typically a 5-10 membered heteroaromatic. Specific exemplary heteroaryl-heteroaryl groups include those in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. In some embodiments, the heteroaromatic ring systems are 5-15 membered heteroaromatic rings, more typically 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In some specific exemplary embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Substituted,” when used to modify a specified group or radical, means that one or more hydrogen atoms of the specified group or radical are each, independently of one another, replaced with the same or different substituent(s). Substituent groups useful for substituting for hydrogens on saturated carbon atoms in the specified group or radical include, but are not limited to —R⁶⁰, halo, —O⁻M⁺, ═O, —OR⁷⁰, —SR⁷⁰, —S⁻M⁺, ═S, —NR⁸⁰R⁸⁰, ═NR⁷⁰, ═N—OR⁷⁰, trihalomethyl, —CF₃, —CN, —OCN, —SCN, —NO, —NO₂, ═N₂, —N₃, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of alkyl, cycloalkyl, heteroalkyl, cycloheteroalkyl, aryl, arylalkyl, heteroaryl and heteroarylalkyl; each R⁷⁰ is independently hydrogen or R⁶⁰; each R⁸⁰ is independently R⁷⁰ or alternatively, the two R⁸⁰'s, taken together with the nitrogen atom to which they are bonded, form a 5-, 6- or 7-membered cycloheteroalkyl which may optionally include from 1 to 4 of the same or different additional heteroatoms selected from the group consisting of O, N and S; and each M⁺ is a counter ion with a positive charge, for example, a positive charge independently selected from K⁺, Na⁺, +N(R⁶⁰)₄, and Li⁺, or two of M⁺, combine to form a divalent counterion, for example a divalent counterion selected from Ca²⁺, Mg²⁺, and Ba²⁺. As specific examples, —NR⁸⁰R⁸⁰ is meant to include —NH₂, —NH-alkyl, N-pyrrolidinyl and N-morpholinyl.

Similarly, substituent groups useful for substituting for hydrogens on unsaturated carbon atoms in the specified group or radical include, but are not limited to, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)₂R⁷⁰, —S(O)₂O⁻M⁺, —S(O)₂OR⁷⁰, —O S(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰) NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR^{7 0}C(S)R⁷⁰, —NR⁷⁰C(O)O⁻M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups, other than R^{p}, useful for substituting for hydrogens on nitrogen atoms in heteroalkyl and cycloheteroalkyl groups include, but are not limited to, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂ O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Substituent groups from the above lists useful for substituting other groups or atoms specified as “substituted” will be apparent to those of skill in the art.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

The term “pharmaceutically acceptable salt” means a salt which is acceptable for administration to a subject, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a subject. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder.

### Methods of Treatment

This disclosure provides, among other things, a method for treating sickle cell disease, particularly to reduce immunological responses that manifest in the disease. In some embodiments, the subject may exhibiting one or more of the following symptoms: anemia, sickle cell crisis, vaso-occlusive crisis, splenic sequestration crisis, splenic sequestration crises, acute chest syndrome, acute chest syndrome, aplastic crisis, haemolytic crisis, dactylitis, pneumonia, respiratory infection, bone-marrow embolisation, or atelectasis.

Sickle cell disease (SCD) is a group of blood disorders typically inherited. The most common type is known as sickle cell anemia, which results in an abnormality in the oxygen-carrying protein haemoglobin found in red blood cells. This leads to a rigid, sickle-like shape under certain circumstances. Problems in sickle cell disease typically begin around 5 to 6 months of age and a number of health problems may develop, such as attacks of pain (known as a sickle cell crisis), anemia, swelling in the hands and feet, bacterial infections and stroke. Long-term pain may develop as people get older.

Sickle cell disease occurs when a person inherits two abnormal copies of the β-globin gene (HBB) that makes haemoglobin, one from each parent. That gene occurs in chromosome 11. Several subtypes exist, depending on the exact mutation in each haemoglobin gene. An attack can be set off by temperature changes, stress, dehydration, and high altitude.

The care of people with sickle cell disease may include infection prevention with vaccination and antibiotics, high fluid intake, folic acid supplementation, and pain medication. Other measures may include blood transfusion and the medication hydroxycarbamide (hydroxyurea). A small percentage of people can be cured by a transplant of bone marrow cells. Patients with sickle cell disease may exhibit the following symptoms:

Sickle cell crisis: The terms “sickle cell crisis” or “sickling crisis” may be used to describe several independent acute conditions occurring in subjects with SCD, which results in anaemia and crises that could be of many types, including the vaso-occlusive crisis, aplastic crisis, splenic sequestration crisis, haemolytic crisis, and others. Most episodes of sickle cell crises last between five and seven days. Although infection, dehydration, and acidosis (all of which favor sickling) can act as triggers, in most instances, no predisposing cause is identified.

Vaso-occlusive crisis: The vaso-occlusive crisis is caused by sickle-shaped red blood cells that obstruct capillaries and restrict blood flow to an organ, resulting in ischaemia, pain, necrosis, and often organ damage. The frequency, severity, and duration of these crises vary considerably. Painful crises are treated with hydration, analgesics, and blood transfusion; pain management requires opioid drug administration at regular intervals until the crisis has settled. For milder crises, a subgroup of subjects manages on nonsteroidal anti-inflammatory drugs such as diclofenac or naproxen. For more severe crises, most subjects require in-subject management for intravenous opioids; subject-controlled analgesia devices are commonly used in this setting. Vaso-occlusive crisis involving organs such as the penis or lungs are considered an emergency and treated with red blood cell transfusions. Incentive spirometry, a technique to encourage deep breathing to minimise the development of atelectasis, is recommended.

Splenic sequestration crisis: The spleen is frequently affected in sickle cell disease, as the sickle-shaped red blood cells cause narrowing of blood vessels and reduced function in clearing the defective cells. It is usually infarcted before the end of childhood in individuals with sickle cell anaemia. This spleen damage increases the risk of infection from encapsulated organisms; preventive antibiotics and vaccinations are recommended for those lacking proper spleen function.

Splenic sequestration crises are acute, painful enlargements of the spleen, caused by intrasplenic trapping of red cells and resulting in a precipitous fall in haemoglobin levels with the potential for hypovolemic shock. Sequestration crises are considered an emergency. If not treated, subjects may die within 1-2 hours due to circulatory failure. Management is supportive, sometimes with blood transfusion. These crises are transient; they continue for 3-4 hours and may last for one day.

Acute chest syndrome: Acute chest syndrome is defined by at least two of these signs or symptoms: chest pain, fever, pulmonary infiltrate or focal abnormality, respiratory symptoms, or hypoxemia. It is the second-most common complication and it accounts for about 25% of deaths in subjects with SCD. Most cases present with vaso-occlusive crises, and then develop acute chest syndrome. Nevertheless, about 80% of people have vaso-occlusive crises during acute chest syndrome.

Aplastic crisis: Aplastic crises are instances of an acute worsening of the subject's baseline anaemia, producing pale appearance, fast heart rate, and fatigue. This crisis is normally triggered by parvovirus B19, which directly affects production of red blood cells by invading the red cell precursors and multiplying in and destroying them. Parvovirus infection almost completely prevents red blood cell production for two to three days. In normal individuals, this is of little consequence, but the shortened red cell life of SCD subjects results in an abrupt, life-threatening situation. Reticulocyte counts drop dramatically during the disease (causing reticulocytopenia), and the rapid turnover of red cells leads to the drop in haemoglobin. This crisis takes 4 to 7 days to disappear. Most subjects can be managed supportively; some need a blood transfusion.

Haemolytic crisis: Haemolytic crises are acute accelerated drops in haemoglobin level. The red blood cells break down at a faster rate. This is particularly common in people with coexistent G6PD deficiency. Another influence of hemolytic crises in Sickle Cell Disease is oxidative stress on the erythrocytes, leukocytes, and platelets. When there is not enough red blood cell production in the bone marrow, the oxygen that the body receives, processes, and transports is unbalanced with the body's antioxidants. There is an imbalance in the oxygen reactive species in the cells, which leads to more production of red blood cells that are not properly oxygenated or formed. Oxidative stress may lead to anemia because of the imbalance of oxygen in the tissue. Management is supportive, sometimes with blood transfusions.

In addition, one of the earliest clinical manifestations is dactylitis, presenting as early as six months of age, and may occur in children with sickle cell trait. The crisis can last up to a month. Given that pneumonia and sickling in the lung can both produce symptoms of acute chest syndrome, the subject is treated for both conditions. It can be triggered by painful crisis, respiratory infection, bone-marrow embolisation, or possibly by atelectasis, opiate administration, or surgery. Hematopoietic ulcers may also occur.

In certain embodiments, provided herein is a method of treating a subject having or suspected of having SCD, the method comprising administering to the subject 100 to 300 mg of fostamatinib once daily (QD), or 100 to 200 mg of fostamatinib or a pharmaceutically acceptable salt thereof twice daily (BID). In certain embodiments, the amount of fostamatinib administered is 100 rug BID. In yet other certain embodiments, the amount of fostamatinib administered is 150 mg BID. In further certain embodiments, the amount of fostamatinib administered is 200 mg BID. In certain embodiments, fostamatinib is first administered at 100 mg BID for one, two, three or four weeks, followed by administration of fostamatinib at 150 mg BID. In certain embodiments, fostamatinib is administered at 100 mg BID for one, two, three or four weeks, followed by administration of fostamatinib a 150 mg BID for one, two, three or four weeks. In certain embodiments, fostamatinib is administered at 100 mg BID for two weeks, followed by administration of fostamatinib at 150 mg BID for four weeks. In certain embodiments, fostamatinib is administered QD, such as 100, 150, 200 or 300 mg QD. In certain embodiments, fostamatinib is administered at 100 mg QD. In certain embodiments, fostamatinib is administered at 150 mg QD. In certain embodiments, fostamatinib is first administered at 150 mg BID for one, two, three or four weeks, followed by administration of fostamatinib at 200 mg BID.

“Neutropenia” is a condition wherein a subject exhibits an abnormally low neutrophil count. For example, less than 1×10⁹/L, absolute neutrophil count or less than 1 0.5×10⁹/L absolute neutrophil count. In one embodiment, a subject may exhibit neutropenia following administration of fostamatinib. In certain embodiments a subject that is administered hydroxy urea and fostamatinib may exhibit neutropenia. In certain embodiments, the present method includes measuring neutrophil count, and when the absolute neutrophil count for a subject is less than 1×10⁹/L, pausing dosing of fostamatinib, until the absolute neutrophil count rises, such as to absolute neutrophil count of 1.5×10⁹/L. Once the absolute neutrophil count rises, fostamatinib dosing can be resumed at the same or a lowered dosage. In one embodiment, if neutropenia occurred after dosing 200 mg BID, dosing is resumed at 150 mg BID following absolute neutrophil count recovery. In one embodiment, if neutropenia occurred after dosing 150 mg BID, dosing is resumed at 100 mg BID following absolute neutrophil count recovery. In one embodiment, dosage reduction can be accomplished by shifting from a BID dosing schedule to a QD dosing schedule. In one embodiment, the dosage is reduced without neutropenia, such as to avoid neutropenia from occurring.

In certain embodiments, the administration of fostamatinib is maintained at 100 mg BID in the event the subject experiences an adverse reaction, such as hypertension, hepatoxicity, diarrhea, neutropenia or other Grade 3 adverse events. In certain embodiments, the administration of fostamatinib is maintained at 100 mg BID in the event the subject experiences neutropenia. In yet certain embodiments, the administration of fostamatinib is maintained at 100 mg BID in the event the subject experiences an adverse reaction, such as: (i) hypertension as evidenced by systolic reading between 130-139 mm Hg or diastolic between 80-89 mmHg or systolic reading of at least 140 mm Hg or diastolic reading of at least 90 mmHg; (ii) hepatoxicity as evidenced by increase in AST/ALT by 2.5× from pre-treatment level or level at the time of enrollment but <3× upper limit of normal (ULN) or increase in AST/ALT to >3× upper limit of normal to ≤5×ULN and total BL (bilirubin) greater than 2× upper limit of normal (ULN); (iii) Grade 3 diarrhea; (iv) neutropenia as evidenced by decrease of absolute neutrophil count (ANC) to less than 1×10⁹/L; or (v) other Grade 3 adverse events.

In one embodiment, treatment with fostamatinib reduces events in SCD pathophysiology, such as hemolysis and ischemia-reperfusion injury, which induce innate immune dysregulation and facilitate “immunothrombosis” which leads to arterial and venous thrombosis. See, Hebbel R P, Belcher J D, Vercellotti G M. The multifaceted role of ischemia/reperfusion in sickle cell anemia. J Clin Invest 2020; 130(3): 1062-72, and Conran N, De Paula E V. Thromboinflammatory mechanisms in sickle cell disease—challenging the hemostatic balance. Haematologica 2020. Neutrophils can enable immunothrombosis via their ability to express neutrophil extracellular traps (NETs), reactive oxygen species (ROS), and neutrophil serine proteases, all of which can independently, or collectively, upregulate prothrombotic and proinflammatory processes. Dysregulated NET release is a distinct phenomenon associated with SCD pathophysiology observed in both patients with SCD experiencing acute VOC and in animal models of SCD⁷. In one embodiment treatment with fostamatinib as described herein inhibits NET formation, NET release, or both.

In one embodiment, a subject having SCD or at risk of SCD has increased neutrophil activation and NET formation. Neutrophil activation and NET formation can be assessed by those of skill in the art and further consistent with the methods disclosed in, for example, Chen G, Zhang D, Fuchs T A, Manwani D, Wagner D D, Frenette P S. Heme-induced neutrophil extracellular traps contribute to the pathogenesis of sickle cell disease. Blood 2014; 123(24): 3818-27. Accordingly, in one embodiment disclosed herein, neutrophil activation is assessed before fostamatinib administration, after fostamatinib administration, or both. In one embodiment, administration of fostamatinib as described herein results in reduced NET formation. Without being limited to any particular theory, the present inventors believe that reduced NET formation results in lower risk of thrombosis in subjects having or at risk of having SCD.

In one embodiment, a subject having SCD or at risk of SCD exhibits increased reactive oxygen species (ROS). ROS levels can be assessed by those of skill in the art and further consistent with the methods disclosed in, for example, Antwi-Boasiako C, Dankwah G B, Aryee R, Hayfron-Benjamin C, Donkor E S, Campbell A D. Oxidative Profile of Patients with Sickle Cell Disease. Med Sci (Basel). 2019 Jan. 25; 7(2):17. Accordingly, in one embodiment disclosed herein, change from baseline in intracellular reactive oxidative species (ROS) in red blood cells is assessed at different doses of fostamatinib. In one embodiment, administration of fostamatinib results in reduced ROS formation. Without being limited to any particular theory, the present inventors believe that reduced ROS formation results in lower risks of complications associated with SCD.

In one embodiment, a subject having SCD or at risk of SCD exhibits increased platelet activation. Platelet activation levels can be assessed by those of skill in the art and further consistent with the methods disclosed in, for example, relinger AL 3rd, Jakubowski J A, Brooks J K, Carmichael S L, Berny-Lang M A, Barnard M R, Heeney M M, Michelson A D. Platelet activation and inhibition in sickle cell disease (pains) study. Platelets. 2014; 25(1):27-35. Accordingly, in one embodiment disclosed herein, platelet activation is assessed at different doses of fostamatinib. In one embodiment, administration of fostamatinib results in reduced platelet activation. Without being limited to any particular theory, the present inventors believe that reduced platelet activation achieved via fostamatinib treatment results in lower risks of complications associated with SCD.

In one embodiment, a subject having SCD or at risk of SCD exhibits increased increased expression of high mobility group box 1 (HMGB1), a critical mediator of venous thrombosis whose plasma levels are elevated in SCD can amplify vascular injury. HMGB1 levels can be assessed by those of skill in the art and further consistent with the methods disclosed in, for example, Xu H, Wandersee N J, Guo Y, et al. Sickle cell disease increases high mobility group box 1: a novel mechanism of inflammation. Blood 2014; 124(26): 3978-81 and Stark K, Philippi V, Stockhausen S, et al. Disulfide HMGB1 derived from platelets coordinates venous thrombosis in mice. Blood 2016; 128(20): 2435-49. Accordingly, in one embodiment disclosed herein, HMGB1 level is assessed following administration of different doses of fostamatinib. In one embodiment, administration of fostamatinib results in reduced HMGB1. Without being limited to any particular theory, the present inventors believe that reduced HMGB1 levels achieved via fostamatinib treatment results in lower risks of complications associated with SCD.

In one embodiment, a subject having SCD or at risk of SCD exhibits increased increased sickling of cells or an increased rate of sickling. Sickling is believed to be correlated with increased risks of SCD complications. See, Brunson A, Lei A, Rosenberg A S, White R H, Keegan T, Wun T. Increased incidence of VTE in sickle cell disease patients: risk factors, recurrence and impact on mortality. Br J Haematol 2017; 178(2): 319-26. In one embodiment administration of fostamatinib to a subject having SCD or at risk of SCD results in increased sickling time, reduced percentage of sickled cells, or both. Sickle cell percentage and kinetics of sickling can be assessed as is known to those of skill in the art and further consistent with Rab MAE, van Oirschot B A, Bos J, Merkx T H, van Wesel A C W, Abdulmalik O, Safo M K, Versluijs B A, Houwing M E, Cnossen M H, Riedl J, Schutgens R E G, Pasterkamp G, Bartels M, van Beers E J, van Wijk R. Rapid and reproducible characterization of sickling during automated deoxygenation in sickle cell disease patients. Am J Hematol. 2019 May; 94(5):575-584. In one embodiment, administration of fostamatinib as described herein results in lower percentages of sickling, slower sickling, or both. In one embodiment, red blood cell (RBC) membrane band3 tyrosine phosphorylation is indicative of sickling and anti-sickling effects exerted by fostamatinib treatment.

The administering can be done any convenient way. For example, the administration may be systemic, e.g., orally (via injection of tablet, pill or liquid) or intravenously (by injection or via a drip, for example). In other embodiments, the administering can be done by pulmonary administration, e.g., using an inhaler or nebulizer.

### Compounds

Compounds that find use in the invention are generally 2,4-pyrimidinediamine compounds according to structural formula (I):

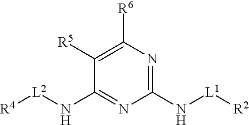

including salts (e.g., pharmaceutically acceptable salts), hydrates, solvates and N-oxides thereof, wherein:
L¹ and L² are each, independently of one another, selected from the group consisting of a direct bond and a linker;
R² and R⁴ are as described in the following embodiments and examples;
R⁵ is selected from the group consisting of R⁶, (C1-C6) alkyl optionally substituted with one or more of the same or different R⁸ groups, (C1-C4) alkanyl optionally substituted with one or more of the same or different R⁸ groups, (C2-C4) alkenyl optionally substituted with one or more of the same or different R⁸ groups and (C2-C4) alkynyl optionally substituted with one or more of the same or different R⁸ groups;
each R⁶ independently is selected from the group consisting of hydrogen, an electronegative group, —OR^{d1}, —SR^{d1}, (C1-C3) haloalkyloxy, (C1-C3) perhaloalkyloxy, —NR^{c}R^{c}, halogen, (C1-C3) haloalkyl, (C1-C3)
   perhaloalkyl, —CF₃, —CH₂CF₃, —CF₂CF₃, —CN, —NC, —OCN, —SCN, —NO, —NO₂, —N₃, —S(O)R^{d1}, —S(O)₂R^{d1}, —S(O)₂OR^{d1}, —S(O)NR^{c}R^{c}, —S(O)₂NR^{c}R^{c}, —OS(O)R^{d1}, —OS(O)₂R^{d1}, —OS(O)₂OR^{d1}, —OS(O)NR^{c}R^{c}, —OS(O)₂NR^{c}R^{c}, —C(O)R^{d1}, —C(O)OR^{d1}, —C(O)NR^{c}R^{c}, —C(NH)NR^{c}R^{c}, —OC(O)R^{d1}, —SC(O)R^{d1}, —OC(O)OR^{d1}, —SC(O)OR^{d1}, —OC(O)NR^{c}R^{c}, —SC(O)NR^{c}R^{c}, —OC(NH)NR^{c}R^{c}, —SC(NH) NR^{c}R^{c}, —[NHC(O)]ₙR^{d1}, —[NHC(O)]ₙOR^{d1}, —[NHC(O)]ₙNR^{c}R^{c} and —[NHC(NH)]ₙNR^{c}R^{c}, (C5-C10) aryl optionally substituted with one or more of the same or different R⁸ groups, phenyl optionally substituted with one or more of the same or different R⁸ groups, (C6-C16) arylalkyl optionally substituted with one or more of the same or different R⁸ groups, 5-10 membered heteroaryl optionally substituted with one or more of the same or different R⁸ groups and 6-16 membered heteroarylalkyl optionally substituted with one or more of the same or different R⁸ groups;
R⁸ is selected from the group consisting of R^{a}, R^{b}, R^{a} substituted with one or more of the same or different R^{a} or R^{b}, —OR^{a} substituted with one or more of the same or different R^{a} or R^{b}, —B(OR^{a})₂, —B(NR^{c}R^{c})₂, —(CH₂)ₘ—R^{b}, —(CHR^{a})ₘ—R^{b}, —O—(CH₂)ₘ—R^{b}, —S—(CH₂)ₘ—R^{b}, —O—CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})ₘ—R^{b}, —C(O)NH—(CHR^{a})ₘ—R^{b}, —NH—(CH₂)ₘ—R^{b}, —NH—(CHR^{a})ₘ—R^{b}, —NH[(CH₂)ₘR^{b}], —N[(CH₂)ₘR^{b}]₂, —NH—C(O)—NH—(CH₂)ₘ—R^{b}, —NH—C(O)—(CH₂)ₘ—CHR^{b}R^{b} and —NH—(CH₂)ₘ—C(O)—NH—(CH₂)ₘ—R^{b};
each R^{a} is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b} is a suitable group independently selected from the group consisting of ═O, —OR^{d1}, (C1-C3) haloalkyloxy, —OCF₃, ═S, —SR^{d1}, ═NR^{d1}, ═NOR^{d1}, —NR^{c}R^{c}, halogen, —CF₃, —CN, —NC, —OCN, —SCN, —NO, —NO₂, ═N₂, —N₃, —S(O)R^{d1}, —S(O)₂R^{d1}, —S(O)₂OR^{d1}, —S(O)NR^{c}R^{c}, —S(O)₂NR^{c}R^{c}, —OS(O)R^{d1}, —OS(O)₂R^{d1}, —OS(O)₂OR^{d1}, —OS(O)₂NR^{c}R^{c}, —C(O)R^{d1}, —C(O)OR^{d1}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d1}, —OC(O)OR^{d1}, —OC(O)NR^{c}R^{c}, —OC(NH)NR^{c}R^{c}, —O C(NR^{a})NR^{c}R^{c}, —[NHC(O)]ₙR^{d1}, —[NR^{a}C(O)]ₙR^{d1}, —[NHC(O)]ₙOR^{d1}, —[NR^{a}C(O)]ₙOR^{d1}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c} is independently R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which is optionally substituted with one or more of the same or different R^{a} or suitable R^{b} groups;
each R^{d1} is independently R^{a};
each m is independently an integer from 1 to 3; and
each n is independently an integer from 0 to 3.

In the compounds of structural formula (T), L¹ and L² represent, independently of one another, a direct bond or a linker. Thus, as will be appreciated by skilled artisans, the substituents R² and/or R⁴ may be bonded either directly to their respective nitrogen atoms or, alternatively, spaced away from their respective nitrogen atoms by way of a linker. The identity of the linker is not critical and typical suitable linkers include, but are not limited to, (C1-C6) alkyldiyls, (C1-C6) alkanos and (C1-C6) heteroalkyldiyls, each of which may be optionally substituted with one or more of the same or different R⁸ groups, where R⁸ is as previously defined for structural formula (I). In a specific embodiment, L¹ and L² are each, independently of one another, selected from the group consisting of a direct bond, (C1-C3) alkyldiyl optionally substituted with one or more of the same or different R^{a}, suitable R^{b} or R⁹ groups and 1-3 membered heteroalkyldiyl optionally substituted with one or more of the same or different R^{a}, suitable R^{b} or R⁹ groups, wherein R⁹ is selected from the group consisting of (C1-C3) alkyl, —OR^{a}, —C(O)OR^{a}, (C5-C10) aryl optionally substituted with one or more of the same or different halogens, phenyl optionally substituted with one or more of the same or different halogens, 5-10 membered heteroaryl optionally substituted with one or more of the same or different halogens and 6 membered heteroaryl optionally substituted with one or more of the same or different halogens; and R^{a} and R^{b} are as previously defined for structural formula (I). Specific R⁹ groups that may be used to substitute L¹ and L² include —OR^{a}, —C(O)OR^{a}, phenyl, halophenyl and 4-halophenyl, wherein R^{a} is as previously defined for structural formula (I).

In certain embodiments, L¹ and L² are each, independently of one another, selected from the group consisting of methano, ethano and propano, each of which may be optionally monosubstituted with an R⁹ group, where R⁹ is as previously defined above.

In certain embodiments, specific R^{a} groups that may be included in R⁹ groups are selected from the group consisting of hydrogen, (C1-C6) alkyl, phenyl and benzyl.

In certain embodiments, L¹ and L² are each a direct bond such that the 2,4-pyrimidinediamine compounds of the invention are compounds according to structural formula (Ia):

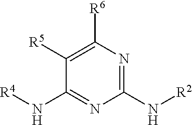

including salts, hydrates, solvates and N-oxides thereof, wherein R², R⁴, R⁵ and R⁶ are as previously defined for structural formula (I). Additional specific embodiments of the 2,4-pyrimidinediamine compounds of the invention are described below.

In certain embodiments of the compounds of structural formula (I) and (Ta), L¹, L², R⁵, R⁶, R⁸, R^{a}, R^{b}, R^{c}, R^{d1}, n and n are as previously defined, R² is

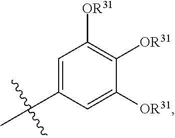

wherein each R³¹, independently of the others, is methyl or (C1-C6) alkyl and R⁴ is

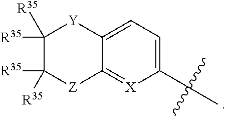

X is selected from the group consisting of N and CH, Y is selected from the group consisting of O, S, SO, SO₂, SONR³⁶, NH, NR³⁵ and NR³⁷, Z is selected from the group consisting of O, S, SO, SO₂, SONR³⁶, NH, NR³⁵ and NR³⁷. Each R³⁵ is, independently of the others, selected from the group consisting of hydrogen and R⁸, or, alternatively, two R³⁵ bonded to the same carbon atom are taken together to form an oxo (═O), NH or NR³⁸ group and the other two R³⁵ are each, independently of one another, selected from the group consisting of hydrogen and R⁸. Each R³⁶ is independently selected from the group consisting of hydrogen and (C1-C6) alkyl. Each R³⁷ is independently selected from the group consisting of hydrogen and a progroup. R³⁸ is selected from the group consisting of (C1-C6) alkyl and (C5-C14) aryl.

In particular, Y is O, Z is NH and X is N. R⁵ can be halogen and R⁶ is a hydrogen.

In certain embodiments of the compounds of structural formula (I) and (Ta), L¹, L², R⁵, R⁶, R⁸, R^{a}, R^{b}, R^{c}, R^{d1}, m, n, R³⁵, R³⁶, R³⁷, R³⁸, X, Y and Z are as previously defined, R² is

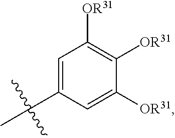

wherein each R³¹, independently of the others, is methyl or (C1-C6) alkyl and R⁴ is

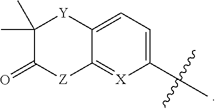

In particular, Y is O, Z is NH and X is N. R⁵ can be halogen and R⁶ is a hydrogen. In one particular aspect, Y is O, Z is NH, X is N and each R³¹ is methyl.

In certain embodiments of the compounds of structural formula (I) and (Ta), L¹, L², R⁶, R⁶, R⁸, R^{a}, R^{b}, R^{c}, R^{d}, in, n, R³¹, R³⁵, R³⁶, R³⁷, R³⁸, X, Y and Z are as previously defined, R² is

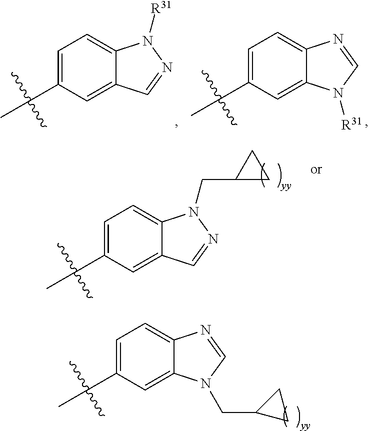

and R⁴ is

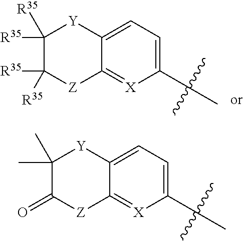

and yy is 1-6. In particular, Y is O, Z is NH and X is N. R⁵ can be halogen and R⁶ is a hydrogen.

In certain embodiments of the compounds of structural formula (I) and (Ta), L¹, L², R⁵, R⁶, R⁸, R^{a}, R^{b}, R^{c}, R^{d1}, m, n, R³⁵, R³⁶, R³⁷, R³⁸, X, Y and Z are as previously defined, R² is

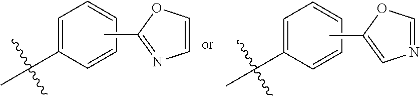

### and R⁴ is

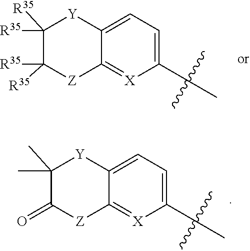

Substitution about the R² phenyl ring can be at the 2, 3, 4, 5 or 6 positions. In particular, Y is O, Z is NH and X is N. R⁵ can be halogen and R⁶ is a hydrogen.

In certain embodiments of the compounds of structural formula (I) and (Ia), L¹, L², R⁵, R⁶, R⁸, R^{a}, R^{b}, R^{c}, R^{d1}, m, n, R³⁵, R³⁶, R³⁷, R³⁸, X, Y and Z are as previously defined, R² is a phenyl group disubstituted with two R^{b} groups and R⁴ is

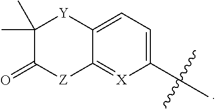

Substitution about the R² phenyl ring can be at the 2,3, 2,4, 2, 5, 2,6, 3,4, 3,5, 3,6, 4,5, 4,6 or 5,6 positions, with the proviso that the following compounds are not included:
N4-(2,2-Dimethyl-3-oxo-4H-5-pyrid[1,4]oxazin-6-yl)-N2-(3-chloro-4-methoxyphenyl)-5-fluoro-2,4-pyrimidinediamine;
N4-(2,2-Dimethyl-3-oxo-4H-5-pyrid[1,4]oxazin-6-yl)-N2-(3,5-dimethoxyphenyl)-5-fluoro-2,4-pyrimidinediamine;
N2-(3,4-Dichlorophenyl)-N4-(2,2-dimethyl-3-oxo-4H-5-pyrid[1,4]oxazin-6-yl)-5-fluoro-2,4-pyrimidinediamine;
N4-(2,2-dimethyl-3-oxo-4H-5-pyrid[1,4]oxazin-6-yl)-N2-(3-fluoro-4-methoxyphenyl)-5-fluoro-2,4-pyrimidinediamine;
N2-(3,5-Dichlorophenyl)-N4-(2,2-dimethyl-3-oxo-4H-5-pyrid[1,4]oxazin-6-yl)-5-fluoro-2,4-pyrimidinediamine; and
N2-(3-Chloro-4-trifluoromethoxyphenyl)-N4-(2,2-dimethyl-3-oxo-4H-5-pyrid[1,4]oxazin-6-yl)-5-fluoro-2,4-pyrimidinediamine.

In particular, Y is O, Z is NH and X is N. R⁵ can be halogen and R⁶ is a hydrogen. In certain aspects, each R^{b} independently is selected from (C1-C6) alkoxy, (C1-16) alkyl, (C1-C6) perhaloalkyls, halogens, carboxylic acid, carboxylic ester, carboxamides, sulfonamides and imidazoles.

In certain embodiments of the compounds of structural formula (I) and (Ia), L¹, L², R⁵, R⁶, R⁸, R^{a}, R^{b}, R^{c}, R^{d1}, n, n, R³⁵, R³⁶, R³⁷, R³⁸, X, Y and Z are as previously defined, R² is a phenyl group trisubstituted with three R^{b} groups and R⁴ is

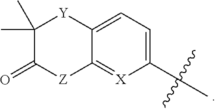

Substitution about the R² phenyl ring can be at the 2,3,4, 2,3,5, 2,3,6, 2,4,5, 2,4,6, 2,5,6, 3,4,5, 3,4,6, 3,5,6, or 4,5,6 positions, with the proviso that the following compounds are not included:
N2-(3-Chloro-4-methoxy-5-methylphenyl)-N4-(2,2-dimethyl-3-oxo-4H-5-pyrid[1,4]oxazin-6-yl)-5-fluoro-2,4-pyrimidinediamine;
N2-(3-Chloro-4-hydroxy-5-methylphenyl)-N4-(2,2-dimethyl-3-oxo-4H-5-pyrid[1,4]oxazin-6-yl)-5-fluoro-2,4-pyrimidinediamine; and
N2-(3,5-Dimethyl-4-methoxyphenyl)-N4-(2,2-dimethyl-3-oxo-4H-5-pyrid[1,4]oxazin-6-yl)-5-fluoro-2,4-pyrimidinediamine.

In particular, Y is O, Z is NH and X is N. R⁵ can be halogen and R⁶ is a hydrogen. In certain aspects, each R^{b} independently is selected from (C1-C6) alkoxy, (C1-16) alkyl, (C1-C6) perhaloalkyls, halogens, carboxylic acid, carboxylic esters, carboxamides, sulfonamides

In certain embodiments, R⁵ of the pyrimidine ring is a halogen atom, such as fluorine, and R⁶ of the pyrimidine ring is a hydrogen atom.

In certain embodiments, L¹ and L² are covalent bonds for the above-identified embodiments.

Also specifically described are combinations of the above embodiments.

In certain embodiments, the compound is N4-(2,2-Dimethyl-3-oxo-4H-5-pyrid[1,4]oxazin-6-yl)-5-fluoro-N2-(3,4,5-trimethoxyphenyl)-2,4-pyrimidinediamine, or a pharmaceutically acceptable salt thereof.

Other suitable compounds are described in U.S. Pat. No. 7,122,542, the disclosure of which is incorporated herein by reference.

### Prodrugs of Compounds

Aspects of the present invention include the 2,4-pyrimidinediamine compounds described herein, which may include functional groups that can be masked with progroups to create prodrugs. Such prodrugs are usually, but need not be, pharmacologically inactive until converted into their active drug form. Indeed, many of the active 2,4-pyrimidinediamine compounds include promoieties that are hydrolyzable or otherwise cleavable under conditions of use. Prodrugs of the present invention may include an active component, where the active component is a 2,4-pyrimidinediamine compound as described herein.

In the prodrugs of the invention, any available functional moiety may be masked with a progroup to yield a prodrug. Functional groups within the 2,4-pyrimidinediamine compounds that may be masked with progroups for inclusion in a promoiety include, but are not limited to, amines (primary and secondary), hydroxyls, sulfanyls (thiols), carboxyls, etc. Myriad progroups suitable for masking such functional groups to yield promoieties that are cleavable under the desired conditions of use are known in the art. All of these progroups, alone or in combinations, may be included in the prodrugs of the invention.

The prodrugs generally comprise a biologically active 2,4-pyrimidinediamine compound that is substituted at the nitrogen atom of one or more primary or secondary amine groups with a progroup R^{p} that metabolizes or otherwise transforms under conditions of use to yield the active 2,4-pyrimidinediamine. In some embodiments, the progroup R^{p} is a phosphorous-containing progroup. In some embodiments, the progroup includes a group or moiety that is metabolized under the conditions of use to yield an unstable α-hydroxymethyl, α-aminomethyl or α-thiomethyl intermediate, which then further metabolized in vivo to yield the active 2,4-pyrimidinediamine drug. In some embodiments, the progroup includes an α-hydroxyalkyl, α-aminoalkyl or α-thioalkyl moiety, for example an α-hydroxymethyl, α-aminomethyl, α-thiomethyl moiety, that metabolizes under the conditions of use to yield the active 2,4 pyrimidinediamine drug. For example, in some embodiments the progroup R^{p} is of the formula —CR^{d}R^{d}-AR³, where each R^{d} is, independently of the other, selected from hydrogen, cyano, optionally substituted (C1-C20) alkyl, (C1-C20) perfluoroalkyl, optionally substituted (C7-C30) arylalkyl and optionally substituted 6-30 membered heteroarylalkyl, where each optional substituent is, independently of the others, selected from hydrogen, alkyl, aryl, arylalkyl, heteroaryl and heteroalkyl, or, alternatively, the two R^{d} are taken together with the carbon atom to which they are bonded to form a cycloalkyl containing from 3 to 8 carbon atoms; A is selected from O, S and NR⁵⁰, where R⁵⁰ is selected from hydrogen, alkyl, aryl, arylalkyl, heteroaryl, heteroarylalkyl and cycloheteroalkyl, or alternatively is combined with R³, and, together with the nitrogen to which they are attached, form a three to seven membered ring; and R³ represents a group that can be metabolized in vivo to yield a group of the formula —CR^{d}R^{d}-AH, where R^{d} and A are as previously defined.

The identity of R³ is not critical, provided that it can be metabolized under the desired conditions of use, for example under the acidic conditions found in the stomach and/or by enzymes found in vivo, to yield a group of the formula —CR^{d}R^{d}-AH, where A and R^{d} are as previously defined. Thus, skilled artisans will appreciate that R³ can comprise virtually any known or later-discovered hydroxyl, amine or thiol protecting group. Non-limiting examples of suitable protecting groups can be found, for example, in Protective Groups in Organic Synthesis, Greene & Wuts, 2nd Ed., John Wiley & Sons, New York, 1991 (especially pages 10-142 (alcohols, 277-308 (thiols) and 309-405 (amines) the disclosure of which is incorporated herein by reference).

In a specific embodiment, R³ includes, together with A, an ether, a thioether, a silyl ether, a silyl thioether, an ester, a thioester, an amide, a carbonate, a thiocarbonate, a carbamate, a thiocarbamate, or a urea linkage, —OCH₂SO₃R, where R is hydrogen, alkyl, aryl, arylalkyl or a metal salt (e.g., sodium, lithium, potassium); -GCH₂⁺N(R⁵¹)₃M⁻, where G is absent, —OPO₃—, OSO₃— or —CO₂—, R⁵¹ is hydrogen, alkyl, aryl, arylalkyl, cycloheteroalkyl or cycloheteroalkylalkyl and M- is a counterion, usually a halide ion or the like (acetate, sulfate, phosphate, etc.). Specific exemplary embodiments include, but are not limited to, progroups R^{p} in which R³ is selected from R^{f}, —C(O)R^{f}, —C(O)OR^{f}, —C(O)NR^{f}R^{f} and —SiR^{f}R^{f}R^{f}, where each R^{f} is, independently of the others, selected from hydrogen, optionally substituted lower alkyl, optionally substituted lower heteroalkyl, optionally substituted lower cycloalkyl, optionally substituted lower heterocycloalkyl, optionally substituted (C6-C10) aryl, optionally substituted 5-10 membered heteroaryl, optionally substituted (C7-C18) arylalkyl and optionally substituted 6-18 membered heteroarylalkyl. In a specific embodiment, each R^{f} is the same.

The identity of the progroup(s) R^{p} can be selected to tailor the water-solubility and other properties of the underlying active 2,4-pyrimidinediamine compound to be optimized for a particular mode of administration. It can also be selected to provide for removal at specified organs and/or tissues within the body, such as, for example, in the digestive tract, in blood and/or serum, or via enzymes residing in specific organs, such as the liver.

In some embodiments, progroups R^{p} that are phosphorous-containing progroups include phosphate moieties that can be cleaved in vitro by enzymes such as esterases, lipases and/or phosphatases. Such enzymes are prevalent throughout the body, residing in, for example, the stomach and digestive tract, blood and/or serum, and in virtually all tissues and organs. Such phosphate-containing progroups R^{p} will generally increase the water-solubility of the underlying active 2,4-pyrimidinediamine compound, making such phosphate-containing prodrugs ideally suited for modes of administration where water-solubility is desirable, such as, for example, oral, buccal, intravenous, intramuscular and ocular modes of administration.

In some embodiments, each phosphate-containing progroup R^{p} in the prodrug is of the formula —(CR^{d}R^{d})_{y}—O—P(O)(OH)(OH), or a salt thereof, wherein R^{d} is as previously defined and y is an integer ranging from 1 to 3, typically 1 or 2. In one specific embodiment, each R^{d} is, independently of the others, selected from hydrogen, substituted or unsubstituted lower alkyl, substituted or unsubstituted phenyl, substituted or unsubstituted methyl and substituted or unsubstituted benzyl. In another specific embodiment, each R^{d} is, independently of the others, selected from hydrogen and unsubstituted lower alkyl. Specific exemplary phosphate-containing progroups R^{p} include —CH₂—O—P(O)(OH)(OH) and —CH₂CH₂—O—P(O)(OH)(OH) and/or the corresponding salts.

While not intending to be bound by any theory of operation, when y is 1 in the exemplary phosphate-containing progroups R^{p}, it is believed that the phosphate-containing prodrugs are converted in vivo by enzymes such as phosphatases, lipases and/or esterases to the corresponding hydroxymethylamines, which are then further metabolized in vivo by the elimination of formaldehyde to yield the active 2,4-pyrimidinediamine drug compound. The phosphate and formaldehyde metabolic by-products are innocuous.

When y is 2 in the exemplary phosphate-containing prodrugs, it is believed that the prodrugs are metabolized to the active 2,4-pyrimidinediamine drug compound in vivo by elimination of enol phosphate, which further metabolizes to acetaldehyde and phosphate. The phosphate and acetaldehyde metabolic by-products are innocuous.

Skilled artisans will appreciate that certain types of precursors can be converted in vivo to phosphate groups. Such precursors include, by way of example and not limitation, phosphate esters, phosphites and phosphite esters. For example, phosphites can be oxidized in vivo to phosphates. Phosphate esters can be hydrolyzed in vivo to phosphates. Phosphite esters can be oxidized in vivo to phosphate esters, which can in turn be hydrolyzed in vivo to phosphates. As a consequence of the ability of these phosphate precursor groups to convert to phosphates in vivo, the prodrugs can also include progroups that comprise such phosphate precursors. In some embodiments, the phosphate precursor groups may be directly metabolized to the active 2,4-pyrimidinediamine drug, without first being converted into a phosphate prodrug. In other embodiments, prodrugs comprising progroups that include such phosphate precursors are first metabolized into the corresponding phosphate prodrug, which then metabolizes to the active 2,4-pyrimidinediamine drug via a hydroxymethylamine, as discussed above.

In some embodiments, such phosphate precursor groups are phosphate esters. The phosphate esters can be acyclic or cyclic, and can be phosphate triesters or phosphate diesters. Such esters are generally less water-soluble than the corresponding phosphate acid prodrugs and the corresponding active 2,4-pyrimidinediamine compounds, and are therefore typically suitable for modes of delivering prodrugs of active 2,4-pyrimidinediamine compounds where low water-solubility is desired, including, by way of example and not limitation, administration via inhalation. The solubility of the prodrug can be specifically tailored for specific modes of administration by appropriate selection of the number and identity(ies) of the esterifying groups in the phosphate ester.

The mechanism by which the phosphate ester group metabolizes to the corresponding phosphate group can be controlled by appropriate selection of the esterifying moieties. For example, it is well-known that certain esters are acid (or base) labile, generating the corresponding phosphate under the acidic conditions found in the stomach and digestive tract. In instances where it is desirable for the phosphate ester prodrug to metabolize to the corresponding phosphate prodrug in the digestive tract (such as, for example, where the prodrugs are administered orally), phosphate ester progroups that are acid-labile can be selected. Other types of phosphate esters are acid and base stable, being converted into the corresponding phosphates via enzymes found in certain tissues and organs of the body (see, e.g., the various cyclic phosphate esters described in Erion et al., 2004, J. Am. Chem. Soc. 126:5154-5163, incorporated herein by reference). In instances where it is desirable to convert a phosphate ester prodrug into the corresponding phosphate prodrug within a desired target tissue or site within the body, phosphate esters having the desired metabolic properties can be selected.

In some embodiments, each phosphate ester-containing progroup R^{p} in the prodrug is an acyclic phosphate ester of the formula —(CR^{d}R^{d})_{y}—O—P(O)(OH)(OR^{e}) or —(CR^{d}R^{d})_{y}—O—P(O)(OR^{e})(OR^{e}), or a salt thereof, wherein each R^{e} is, independently of the others, selected from substituted or unsubstituted lower alkyl, substituted or unsubstituted (C6-C14) aryl (e.g., phenyl, naphthyl, 4-loweralkoxyphenyl, 4-methoxyphenyl), substituted or unsubstituted (C7-C20) arylalkyl (e.g., benzyl, 1-phenylethan-1-yl, 2-phenylethan-1-yl), —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f} and —Si(R^{d})₃, wherein R^{d}, R^{f} and y are as defined above. In a specific embodiment, each R^{d} is selected from hydrogen and unsubstituted lower alkyl and/or each R^{e} is an unsubstituted lower alkanyl or benzyl. Specific exemplary phosphate ester progroups include, but are not limited to, —CH₂—O—P(O)(OH)(OR^{e}), —CH₂CH₂—O—P(O)(OH)(OR^{e}), —CH₂—O—P(O)(OR^{e})(OR^{e}) and —CH₂CH₂—O—P(O)(OR^{e})(OR^{e}), where R′ is selected from lower alkanyl, i-propyl and t-butyl.

In other embodiments, each phosphate ester-containing progroup R^{p} is a cyclic phosphate ester of the formula

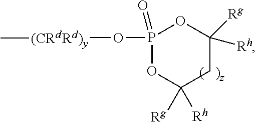

where each R^{g} is, independently of the others, selected from hydrogen and lower alkyl; each R^{h} is, independently of the others, selected from hydrogen, substituted or unsubstituted lower alkyl, substituted or unsubstituted lower cycloheteroalkyl, substituted or unsubstituted (C6-C14) aryl, substituted or unsubstituted (C7-C20) arylalkyl and substituted or unsubstituted 5-14 membered heteroaryl; z is an integer ranging from 0 to 2; and R^{d} and y are as previously defined. In a specific embodiment, each phosphate ester-containing progroup R^{p} is a cyclic phosphate ester of the formula

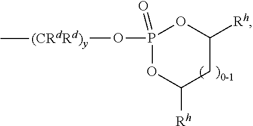

where R^{d}, R^{h} and y are as previously defined.

The mechanism by which cyclic phosphate ester prodrugs including such cyclic phosphate ester progroups metabolize in vivo to the active drug compound depends, in part, on the identity of the R^{h} substitutent. For example, cyclic phosphate ester progroups in which each R^{h} is, independently of the others, selected from hydrogen and lower alkyl are cleaved in vivo by esterases. Thus, in some embodiments, the cyclic phosphate ester progroups are selected such that they are cleavable in vivo by esterases. Specific examples of such cyclic phosphate ester progroups include, but are not limited to, progroups selected from

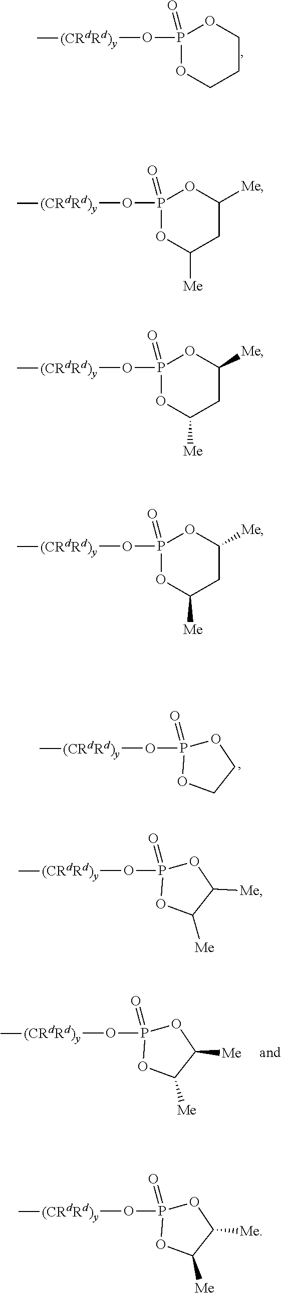

Alternatively, cyclic phosphate ester prodrugs having progroups in which the R^{h} substituents are substituted or unsubstituted aryl, arylalkyl and heteroaryl groups, are not typically cleaved by esterases, but are instead metabolized to the active prodrug by enzymes, such as cytochrome P₄₅₀ enzymes, that reside in the liver. For example, a series of cyclic phosphate ester nucleotide prodrugs that undergo an oxidative cleavage reaction catalyzed by a cytochrome P₄₅₀ enzyme (CYP) expressed predominantly in the liver are described in Erion et al., 2004, J. Am. Chem. Soc. 126:5154-5163. In some embodiments, the cyclic phosphate ester progroups are selected such that they are cleavable by CYP enzymes expressed in the liver. Specific exemplary embodiments of such cyclic phosphate ester-containing progroups R^{p} include, but are not limited to, progroups having the formula

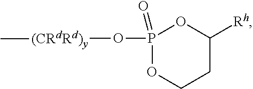

where R^{h} is selected from phenyl, 3-chlorophenyl, 4-pyridyl and 4-methoxyphenyl.

As skilled artisans will appreciate, phosphites and phosphite esters can undergo oxidation in vivo to yield the corresponding phosphate and phosphate ester analogs. Such reactions can be carried out in vivo by, for example, oxidase enzymes, oxoreductase enzymes and other oxidative enzymes. Thus, the phosphorous-containing progroups R^{p} can also include phosphite and phosphite ester analogs of any of the phosphate and phosphate ester progroups described above. In some embodiments the phosphorous-containing progroups R^{p} include, but are not limited to, groups of the formula —(CR^{d}R^{d})_{y}—O—P(OH)(OH), —(CR^{d}R^{d})_{y}—O—P(OH)(OR^{e}) and —(CR^{d}R^{d})_{y}—O—P(OR^{e})(R^{e}), or salts thereof, where R^{d}, R′ and y are as previously defined. Specific exemplary embodiments include groups in which each R^{d} is, independently of the others, selected from hydrogen and unsubstituted lower alkyl and/or each R^{e} is, independently of the others, selected from unsubstituted lower alkanyl and benzyl. Specific exemplary acyclic phosphite and phosphite-ester progroups include, but are not limited to, —CH₂—O—P(OH)(OH), —CH₂CH₂—O—P(OH)(OH), —CH₂—O—P(OH)(OR^{e}), and —CH₂CH₂—O—P(OR^{e})(OR^{e}), where each R^{e} is selected from lower alkanyl, i-propyl and t-butyl. Specific exemplary cyclic phosphite ester prodrugs include phosphite analogs of the above-described cyclic phosphate ester progroups. Conceptually, prodrug compounds including such phosphite and/or phosphite ester progroups can be thought of as prodrugs of the corresponding phosphate and phosphate ester prodrugs.

As mentioned above, it is believed that certain phosphate-containing prodrugs metabolize in vivo through the corresponding hydroxymethylamines. Although these hydroxymethylamines metabolize in vivo to the corresponding active 2,4-pyrimidinediamine compounds, they are stable at pH 7 and can be prepared and administered as hydroxyalkyl-containing prodrugs. In some embodiments, each hydroxyalkyl-containing progroup R^{p} of such prodrugs is of the formula —CR^{d}R^{d}—OH, where R^{d} is as previously defined. A specific exemplary hydroxyalkyl-containing progroup R^{p} is —CH₂OH.

Suitable active 2,4-pyrimidinediamine compounds are described, for example, in U.S. application Ser. No. 10/355,543 filed Jan. 31, 2003 (US2004/0029902A1), international application Serial No. PCT/US03/03022 filed Jan. 31, 2003 (WO 03/063794), U.S. application Ser. No. 10/631,029 filed Jul. 29, 2003, international application Serial No. PCT/US03/24087 (WO2004/014382), U.S. application Ser. No. 10/903,263 filed Jul. 30, 2004 (US2005/0234049), and international application Serial No. PCT/US2004/24716, the disclosures of which are incorporated herein by reference. In such 2,4-pyrimidinediamine compounds, the progroup(s) R^{p} can be attached to any available primary or secondary amine, including, for example, the N2 nitrogen atom of the 2,4-pyrimidinediamine moiety, the N4 nitrogen atom of the 2,4-pyrimidinediamine moiety, and/or a primary or secondary nitrogen atom included in a substituent on the 2,4-pyrimidinediamine compound. The use of phosphate-containing progroups R^{p} is especially useful for 2,4-pyrimidinediamine compounds that exhibit poor water solubility under physiological conditions (for example, solubilities of less than about 10 μg/ml). While not intending to be bound by any theory of operation, it is believed that the phosphate-containing progroups aid the solubility of the underlying active 2,4-pyrimidinediamine compound, which in turn increases its bioavailability when administered orally. It is believed that the phosphate progroups R^{p} are metabolized by phosphatase enzymes found in the digestive tract, permitting uptake of the underlying active drug.

It has been discovered that the water solubility and oral bioavailability of a particular biologically active 2,4-pyrimidinediamine compound, illustrated below (Compound 1), increased dramatically when formulated to include a progroup R^{p} of the formula —CH₂—O—P(O)(OH)₂ at the ring nitrogen atom highlighted with the asterisk (Compound 4):

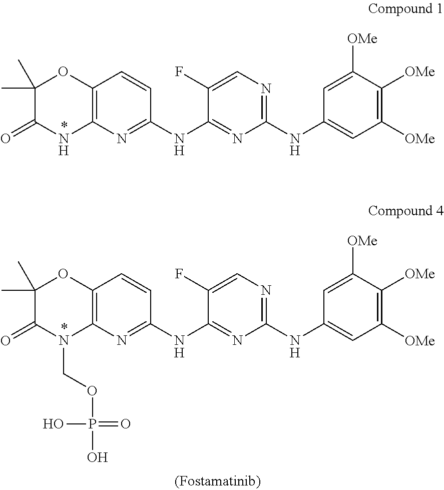 [SMILES: COc1cc(Nc2ncc(F)c(Nc3ccc4c(n3)NC(=O)C(C)(C)O4)n2)cc(OC)c1OC ; COc1cc(Nc2ncc(F)c(Nc3ccc4c(n3)N(COP(=O)(O)O)C(=O)C(C)(C)O4)n2)cc(OC)c1OC]

Significantly, whereas the water solubility of the active drug (Compound 1) is in the range of about 1-2 μg/ml in aqueous buffer under physiological conditions, the solubility of the corresponding phosphate prodrug (Compound 4) is greater than 5 mg/ml under the same conditions, or approximately 2000 times greater. This increased water-solubility allows for better dissolution in the gut, thereby facilitating oral administration. Other active 2,4-pyrimidinediamine compounds having similarly poor water solubilities are expected to exhibit similar increases in water solubility and oral bioavailability when formulated as phosphate prodrugs.

As mentioned above, phosphate ester prodrugs are generally less water-soluble than the corresponding phosphate prodrugs, and are therefore generally useful in applications where low water-solubility is desired, such as, for example, administration via inhalation. The same holds true for the relative water-solubility of phosphite ester and phosphite prodrugs.

In some embodiments, the prodrugs described herein are 2,4-pyrimidinediamine compounds that are substituted at the N4 nitrogen of the 2,4-pyrimidinediamine moiety with a substituted or unsubstituted nitrogen-containing bicyclic ring that includes at least one progroup R^{p} as described herein at one or more of: the nitrogen atom(s) of the bicyclic ring, the N2 nitrogen of the 2,4-pyrimidinediamine moiety and/or the N4 nitrogen of the 2,4-pyrimidinediamine moiety. In a specific illustrative exemplary embodiment, the prodrug is a compound according to structural formula (I):

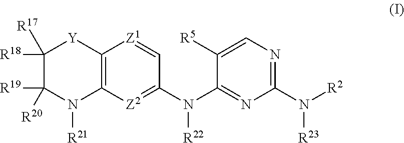

including salts, solvates, hydrates and N-oxides thereof, wherein:
   Y is selected from CH₂, NR²⁴, O, S, S(O) and S(O)₂;
   Z¹ and Z² are each, independently of one another, selected from CH and N;
   R² is an optionally substituted lower alkyl, lower cycloalkyl, lower heteroalkyl, lower cycloheteroalkyl, aryl, phenyl, or heteroaryl group;
   R⁵ is an electronegative group, such as, for example, a halo, fluoro, cyano, nitro, trihalomethyl or trifluoromethyl group;
   R¹⁷ is selected from hydrogen, halogen, fluoro, lower alkyl and methyl or, alternatively, R¹⁷ may be taken together with R¹⁸ to form an oxo (═O) group or, together with the carbon atom to which they are attached, a spirocycle containing from 3 to 7 carbon atoms;
   R¹⁸ is selected from hydrogen, halogen, fluoro, lower alkyl and methyl or, alternatively, R¹⁸ may be taken together with R¹⁷ to form an oxo (═O) group or, together with the carbon atom to which they are attached, a spirocycle containing from 3 to 7 carbon atoms;
   R¹⁹ is selected from hydrogen, lower alkyl, and methyl or, alternatively, R¹⁹ may be taken together with R²⁰ to form an oxo (═O) group or, together with the carbon atom to which they are attached, a spirocycle containing from 3 to 7 carbon atoms;
   R²⁰ is selected from hydrogen, lower alkyl and methyl or, alternatively, R²⁰ may be taken together with R¹⁹ to form an oxo (═O) group or, together with the carbon atom to which they are attached, a spirocycle containing from 3 to 7 carbon atoms;
   R²¹, R²² and R²³ are each, independently of one another, selected from hydrogen and a progroup R^{p} as described herein; and
   R²⁴ is selected from hydrogen, lower alkyl and a progroup R^{p} as described herein, with the proviso that at least one of R²¹, R²², R²³ and R²⁴ must be a progroup R^{p}. In some embodiments, each of R²¹, R²² and R²¹ is one of the specific progroups exemplified above and R²⁴ is hydrogen. In some embodiments R²¹ is one of the specific progroups exemplified above and R²², R²³ and R²⁴ are each hydrogen. In some embodiments, R²¹, R²² and R²³ are each one of the specific progroups exemplified above and R²⁴ is lower alkyl.

In another aspect, the present disclosure provides compositions comprising one or more of the prodrugs described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

In still another aspect, the present disclosure provides intermediates useful for synthesizing the prodrugs described herein. In the case of phosphate- or phosphite-containing prodrugs, the intermediates generally comprise prodrugs in which the oxygen atoms of the phosphate- and/or phosphite-containing progroups are masked with protecting groups that are selectively removable under specified conditions. In some embodiments, the protecting groups are selectively removable under mildly acidic conditions. In some embodiments, the intermediates are phosphate or phosphite esters which are themselves prodrugs that can be metabolized into active 2,4-pyrimidinediamine compounds. In one illustrative embodiment, the intermediates include prodrugs in which each R^{p} progroup is, independently of the others, of the formula —(CR^{d}R^{d})_{y}—O—P(O)(ORⁱ)(ORⁱ), —(CR^{d}R^{d})_{y}—O—P(O)(ORⁱ)(OH), —(CR^{d}R^{d})_{y}—O—P(ORⁱ)(ORⁱ) or —(CR^{d}R^{d})_{y}—O—P(ORⁱ)(OH), where each Rⁱ is, independently of the others, selected from lower unsubstituted alkanyl, substituted or unsubstituted phenyl and substituted or unsubstituted benzyl, and R^{d} and y are as previously defined. In a specific embodiment, the intermediates include phosphate and/or phosphite esters in which each Rⁱ is, independently of the others, selected from lower linear alkanyl, lower branched alkanyl, i-propyl, t-butyl and lower cyclic alkanyl.

In some embodiments, the intermediates comprise an active 2,4-pyrimidinediamine that is substituted at a nitrogen atom of a primary or secondary amine group with a group of the formula —CR^{d}R^{d}-AH, where R^{d} and A are as previously defined.

In yet another aspect, the present disclosure provides methods of synthesizing the intermediates and/or prodrugs described herein. Phosphate-containing prodrugs can be synthesized by reacting an active 2,4-pyrimidinediamine compound with a phosphate ester halide, for example, a phosphate ester halide of the formula X—(CR^{d}R^{d})_{y}—O—P(O)(OR^{j})(OR^{j}) or X—(CR^{d}R^{d})_{y}—O—P(O)(OR^{j})(OH), where each R^{j} is, independently of the others, a selectively removable protecting group; X is a halide, such as, for example, chloride; and R^{d} and y are as previously defined. In some embodiments, each R^{j} is R^{e}, as previously defined. Removal of the selectively removable protecting groups R^{j} yields a phosphate prodrug. In some embodiments each R^{j} is the same and is selected from lower linear alkyl, lower branched alkyl and lower cycloalkyl. In some embodiments, each R^{j} is isopropyl or t-butyl. In embodiments in which mixtures of intermediates are obtained, for example, mixtures of intermediates which contain different numbers of progroups or progroups at different positions on the 2,4-pyrimidinediamine molecule, the desired intermediate can be isolated from the mixture using standard separation and/or isolation techniques (e.g., column chromatography). Alternatively, a desired prodrug can be isolated from a mixture of different prodrugs using standard separation and/or isolation techniques.

Acyclic phosphate ester prodrugs can be obtained in an analogous manner by reacting the active 2,4-pyrimidinediamine with a phosphate ester halide, for example a phosphate ester halide of the formula X—(CR^{d}R^{d})_{y}—O—P(O)(OH)(OR^{e}) or X—(CR^{d}R^{d})_{y}—P(O)(OR^{e})(OR^{e}), where X, R^{d}, y and R^{e} are as previously defined. In this instance, removal of the esterifying groups R^{c} is not necessary.

Acyclic phosphite and phosphite ester prodrugs can be prepared in an analogous manner from the corresponding phosphite ester halides, for example phosphite ester halides of the formula X—(CR^{d}R^{d})_{y}—O—P(OR^{j})(OR^{j}), X—(CR^{d}R^{d})_{y}—O—P(OR^{e})(OH), X—(CR^{d}R^{d})_{y}—O—P(OR^{e})(OR^{e}), where X, R^{d}, y, R^{e} and R^{j} are as previously defined.

Cyclic phosphate ester and phosphite ester prodrugs can be prepared by reacting the active 2,4-pyrimidinediamine compound with the corresponding cyclic phosphate ester or phosphite ester halide, for example, a cyclic phosphate ester halide of the formula

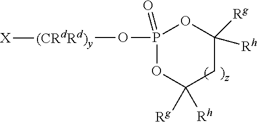

or a cyclic phosphite ester halide of the formula

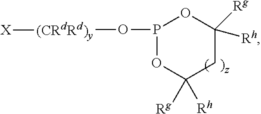

where X, R^{d}, y, z, R^{g} and R^{h} are as previously defined.

Embodiments in which R^{p} is —CR^{d}R^{d}-AR³ can be prepared from the corresponding 2,4-pyrimidinediamine drug using conventional methods. For example, when A is 0, the intermediates can be synthesized by reacting an active 2,4-pyrimidinediamine compound, with an aldehyde or ketone of the formula R^{d}—C(O)—R^{d}, where R^{d} is as previously defined, to yield a corresponding hydroxymethylamine intermediate (where R^{p} is —CR^{d}R^{d}—OH). The hydroxymethylamine intermediate can then be converted into the prodrug using standard techniques. In accordance with the definition of R^{p}, the hydroxymethylamine intermediate is also a prodrug of the invention. For example, other drug substances containing secondary amines have been added to formaldehyde to afford their corresponding isolable hydroxymethylamine adducts, Bansal et al., J. Pharmaceutical Sci. 1981, 70: (8), 850-854; Bansal et al., J. Pharmaceutical Sci. 1981, 70: (8), 855-856; Khan et al., J. Pharmaceutical and Biomedical Analysis 1989, 7 (6), 685-691. Alternatively, hydroxyalkyl-containing prodrugs can be prepared in two steps by first reacting the active 2,4-pyrimidinediamine with a bis-functional electrophile, such as a halide of the formula X¹—CR^{d}R^{d}—X², where X¹ represents a first halide, X² represents a second halide and R^{d} is as previously defined. In a specific exemplary embodiment, the halide is of the formula I—CR^{d}R^{d}—Cl. The unreacted halide is then hydroxylated to yield the hydroxyalkyl-containing prodrug using standard techniques.

Prodrugs in which A is O, S or NR⁵⁰ can be synthesized from corresponding N-methyl phosphate esters. According to this embodiment, the phosphate ester groups can be displaced with a group of the formula R³-AH, where R³ and A are as previously defined, to yield the prodrug, as discussed in further detail below.

In the prodrugs described herein, and in particular in the prodrugs of structural formula (I), R²¹, R²² and R²³ each represent either hydrogen or a progroup R^{p}. Also, R²⁴ represents hydrogen, a lower alkyl or a progroup R^{p}. Thus, the prodrugs can include a single R^{p} progroup, two R^{p} progroups, three R^{p} progroups, or even more R^{p} progroups, depending, in part, on the identity of Y and whether the R² substituent includes any R^{p} progroups. In some embodiments, it is preferred that the prodrugs described herein, and in particular the prodrugs of structural formula (I), include only one R^{p} group. Without intending to be bound by any theory of operation, it is possible that the different R^{p} groups in prodrugs including more than one R^{p} progroup may metabolize at different rates. Prodrugs including a single R^{p} progroup would avoid such differential metabolic kinetics. A specific embodiment of prodrugs according to structural formula (I) that include a single progroup R^{p} are compounds according to structural formula (Ia):

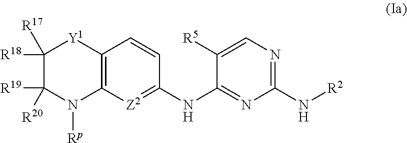

wherein Y¹ is selected from CH₂, NR²⁴, O, S, S(O) and S(O)₂; and Z², R², R⁵, R¹⁷, R¹⁸, R¹⁹, R²⁰, R²⁴ and R^{p} are as previously defined, with the proviso that R² does not include any R^{p} groups.

The identity of any R^{p} progroups present in the prodrugs described herein is not critical for success, provided that it hydrolyzes under the conditions of use to yield the active 2,4-pyrimidinediamine compound. Fostamatinib is a phosphate-containing prodrug according to the structure illustrated below:

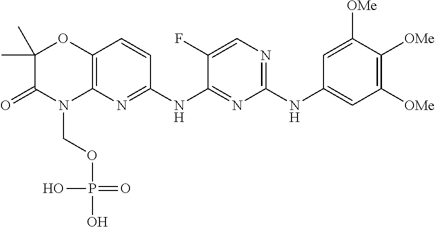 [SMILES: COc1cc(Nc2ncc(F)c(Nc3ccc4c(n3)N(COP(=O)(O)O)C(=O)C(C)(C)O4)n2)cc(OC)c1OC]

metabolizes in vivo to the corresponding active 2,4-pyrimidinediamine compound (Compound 1), illustrated below:

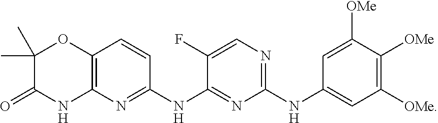 [SMILES: COc1cc(Nc2ncc(F)c(Nc3ccc4c(n3)NC(=O)C(C)(C)O4)n2)cc(OC)c1OC]

While not intending to be bound by any particular theory operation, it is believed that this prodrug metabolizes to active Compound 1 via the corresponding hydroxymethylamine intermediate illustrated below:

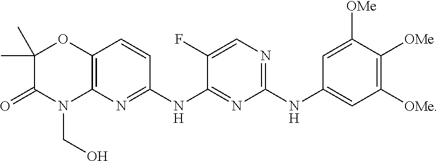 [SMILES: COc1cc(Nc2ncc(F)c(Nc3ccc4c(n3)N(CO)C(=O)C(C)(C)O4)n2)cc(OC)c1OC]

Such hydroxymethylamine compounds are known to be unstable under physiological conditions and various pH ranges where they hydrolyze in vivo to yield formaldehyde and the active drug substance. Based on this observation, it is believed that prodrugs that include hydroxyl “protecting” groups that can be metabolized in vivo, for example by the acidic conditions of the stomach and/or by enzymes present in the digestive tract or other organs and/or tissues or fluids with the body, to yield the hydroxymethylamine intermediate illustrated above will likewise metabolize to the active 2,4 pyrimidinediamine compound.

Moreover, it is expected that the amino and thio analogs of this hydroxymethylamine intermediate, will be similarly unstable at physiological conditions and also hydrolyze in vivo to the active 2,4-pyrimdiendiamine drug. Accordingly, it is also expected that the corresponding amino and thio compounds, as well as compounds in which the α-amino and α-thio groups are masked with “protecting” groups that are removed under physiological conditions of use to yield the α-amino and α-thio groups, will likewise make suitable prodrugs.

Thus, in some embodiments, the progroup(s) R^{p} in the prodrugs of structural formulae (I) and (Ia) are of the formula —CR^{d}R^{d}-A-R³, where each R^{d} is, independently of the other, selected from hydrogen, cyano, —C(O)R^{e}, —C(O)OR^{e}, —C(O)NR^{e}R^{e}, —C(OR^{e})(OR^{e}), optionally substituted (C1-C20) alkyl, (C1-C20) perfluoroalkyl, optionally substituted (C7-C30) arylalkyl and optionally substituted 6-30 membered heteroarylalkyl, where each R^{e} is, independently of the others, selected from hydrogen, alkyl (for example lower alkyl), aryl (for example phenyl or naphthyl, arylalkyl (for example benzyl), heteroaryl and heteroarylalkyl; A is selected from O, S and NR⁵⁰, where R⁵⁰ is selected from R^{d} and cycloalkyl, or, alternatively, is taken together with R³ such that R⁵⁰ and R³, together with nitrogen atom to which they are attached, form a three- to seven-membered ring; and R³ is a group that, together with A, metabolizes under the conditions of use to yield an intermediate group of the formula —CR^{d}R^{d}AH, where R^{d} and A are as previously defined. As mentioned above, compounds of structural formula (I) and (Ia) in which the R^{p} groups are of the formula —CR^{d}R^{d}-AH spontaneously hydrolyze in vivo to yield a biologically active 2,4-pyrimidinediamine compound.

The mechanism by which the R³ group metabolizes to yield intermediate group —CR^{d}R^{d}-A-H is not critical, and can be caused by, for example, hydrolysis under the acidic conditions of the stomach, and/or by enzymes present in the digestive tract and/or tissues or organs of the body. Indeed, the R³ group(s) can be selected to metabolize at a particular site within the body. For example, many esters are cleaved under the acidic conditions found in the stomach. Prodrugs designed to cleave chemically in the stomach to the active 2,4-pyrimidinediamine can employ progroups including such esters. Alternatively, the progroups may be designed to metabolize in the presence of enzymes such as esterases, amidases, lipolases, phosphatases including ATPases and kinase etc., to yield the intermediate group of formula —CR^{d}R^{d}-A-H. Progroups including linkages capable of metabolizing in vivo to yield such an intermediate group are well-known, and include, by way of example and not limitation, ethers, thioethers, silylethers, silylthioethers, esters, thioesters, carbonates, thiocarbonates, carbamates, thiocarbamates, ureas, thioureas, carboxamides, etc. In some instances, a “precursor” group that is oxidized by oxidative enzymes such as, for example, cytochrome P450 of the liver, to a metabolizable group, can be selected.

The identity of the R³ group can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The R³ group can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, targeting-specific transporters, etc. Groups capable of imparting prodrugs with these characteristics are well-known, and are described, for example, in Ettmayer et al., 2004, J. Med. Chem. 47(10:2393-2404), the disclosure of which is incorporated by reference. All of the various groups described in these references can be utilized in the prodrugs described herein.

In some embodiments, R³ is selected from —R^{e}, —C(O)R^{f}, —C(O)NR^{f}R^{f} and —SiR^{f}R^{f}R^{f}, where the R^{f} groups are selected so as to impart the prodrugs with desired bioavailability, cleavage and/or targeting properties. In a specific embodiment, the R^{f} groups are selected to impart the prodrug with higher water-solubility than the underlying active 2,4-pyrimidinediamine drug. Thus, in some embodiments, the R^{f} groups are selected such that they, taken together with the heteroatom or group to which they are bonded, are hydrophilic in character. Such hydrophilic groups can be charged or uncharged, as is well-known in the art. As specific examples, the R^{f} groups may be selected from hydrogen, optionally substituted lower alkyl, optionally substituted lower heteroalkyl, optionally substituted lower cycloalkyl, optionally substituted lower heterocycloalkyl, optionally substituted (C6-C10) aryl, optionally substituted 5-10 membered heteroaryl, optionally substituted (C7-C18) arylalkyl and optionally substituted 6-18 membered heteroarylalkyl. The nature of any present substituents can vary widely, as is known in the art. In some embodiments any present substituents are, independently of one another, selected from R^{b}, defined above.

In a specific embodiment, the progroups on the prodrugs of formula (I) and/or (Ta) are of the formula —CR^{d}R^{d}-A-R³, where R³ is selected from —(CH₂)ᵢ—R^{b}, —C(O)R^{A}, —C(O)—(CH₂)ᵢ—R^{b}, —C(O)O—R^{a} and —C(O)O—(CH₂)ᵢ—R^{b}, where X, R^{A}, R^{b} and R^{d} are as previously defined, and i is an integer ranging from 0 to 6. Specific, non-limiting, examples of exemplary water-solubility increasing progroups include by the way of example and not limitation, hydrophilic groups such as alkyl, arylk, arylalkyl, or cycloheteroalkyl groups substituted with one or more of an amine, alcohol, a carboxylic acid, a phosphorous acid, a sulfoxide, a sugar, an amino acid, a thiol, a polyol, a ether, a thioether and a quaternary amine salt.

One important class of progroups includes progroups that contain a phosphate group, for example, phosphate-containing progroups of the formula —(R^{d}R^{d})_{y}—O—P(O)(OH)₂, where R^{d} is as defined above and y is an integer ranging from 1 to 3, typically 1 or 2. In a specific embodiment, each R^{d} is, independently of the others, selected from hydrogen, substituted or unsubstituted lower alkyl, substituted or unsubstituted (C6-C14) aryl and substituted or unsubstituted (C7-C20) arylalkyl.

While not intending to be bound by any theory of operation, it is believed that such phosphate-containing progroups R^{p} act as substrates for both alkaline and acid phosphatase enzymes, leading to their removal from the prodrugs under physiological conditions of use. As alkaline phosphatases are abundant in the digestive tract of humans, phosphate-containing progroups R^{p} that can be cleaved in the presence of alkaline phosphatases are particularly suitable for formulating phosphate-containing prodrugs intended for oral administration. Specific examples of phosphate-containing progroups R^{p} suitable for use in prodrugs intended for oral administration include, but are not limited to, groups of the formula —(R^{d}R^{d})O—P(O)(OH)₂ in which each R^{d} is, independently of the others, selected from hydrogen and unsubstituted lower alkanyl. Exemplary embodiments of such phosphate-containing progroups include, but are not limited to, —CH₂—O—P(O)(OH)₂ and —CH₂CH₂—O—P(O)(OH)₂.

In some embodiments of such prodrugs, the phosphorous-containing progroup R^{p} comprises a phosphite group. A specific exemplary embodiment of such phosphite-containing prodrugs includes prodrug compounds in which the progroup R^{p} is of the formula —(CR^{d}R^{d})_{y}—O—P(OH)(OH), where R^{d} and y are as previously defined.

In other embodiments of such prodrugs, the phosphorous-containing progroup R^{p} comprises an acyclic phosphate ester or phosphite ester group. Specific exemplary embodiments of such acyclic phosphate ester and phosphite ester prodrugs include progroups R^{p} of the formula —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}) and —(CR^{d}R^{d})_{y}—O—P(OR^{e})₂, where R^{e} is selected from substituted or unsubstituted lower alkyl, substituted or unsubstituted (C6-C14) aryl (e.g., phenyl, naphthyl, 4-lower alkoxyphenyl, 4-methoxyphenyl), substituted or unsubstituted (C7-C20) arylalkyl (e.g., benzyl, 1-phenylethan-1-yl, 2-phenylethan-1-yl), —(CR^{d}R^{d})_{y}—OR^{f}, —(CR^{d}R^{d})—C(O)R^{f}, —(CR^{d}R^{d})—C(O)OR^{f}, —(CR^{d}R^{d})S—C(O)R^{f}, —(CR^{d}R^{d}), —S—C(O)OR^{f}, —(CR^{d}R^{d})_{y}—NH—C(O)R^{f}, —(CR^{d}R^{d})_{y}—NH—C(O)OR^{f} and —Si(R^{d})₃, wherein each R^{f} is, independently of the others, selected from hydrogen, unsubstituted or substituted lower alkyl, substituted or unsubstituted (C6-C14) aryl, and substituted or unsubstituted (C7-C20) arylalkyl, and R^{d} and y are as previously defined.

In still other embodiments, phosphorous-containing prodrugs that include phosphate precursors are prodrugs in which the phosphorous-containing progroup R^{p} comprises a cyclic phosphate ester of the formula

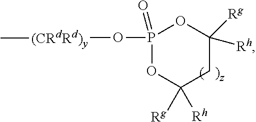

where each R⁹ is, independently of the others, selected from hydrogen and lower alkyl; each R^{h} is, independently of the others, selected from hydrogen, substituted or unsubstituted lower alkyl, substituted or unsubstituted lower cycloheteroalkyl, substituted or unsubstituted (C6-C14) aryl, substituted or unsubstituted (C7-C20) arylalkyl and substituted or unsubstituted 5-14 membered heteroaryl; z is an integer ranging from 0 to 2; and R^{d} and y are as previously defined.

In still other embodiments, phosphorous-containing prodrugs that include phosphate precursors are prodrugs in which the phosphorous-containing progroup R^{p} comprises a cyclic phosphite ester of the formula

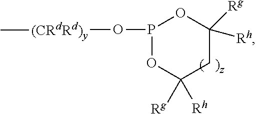

where R⁹, R^{h}, R^{d}, y and z are as previously defined.

In some embodiments, the substituents R^{h} on such cyclic phosphate ester and phosphite ester prodrugs are selected such that the progroup is metabolized in vitro by esterase enzymes. Specific examples of such phosphate ester and phosphite ester progroups include those in which each R^{h} is, independently of the others, selected from hydrogen, lower alkyl, methyl, ethyl and propyl. In some embodiments, such progroups are selected from

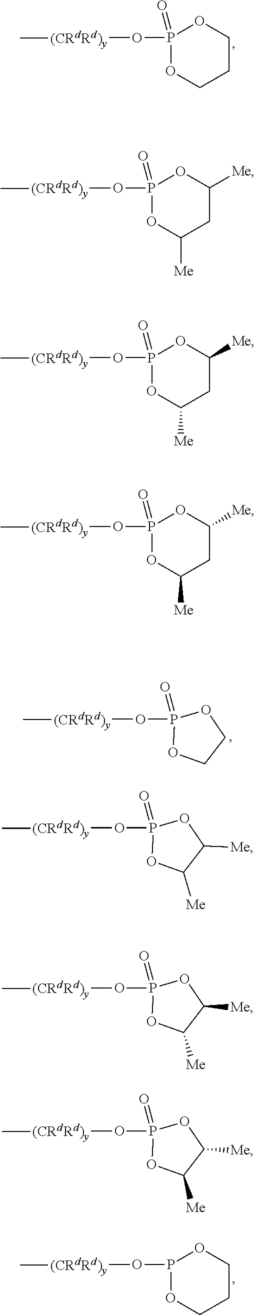

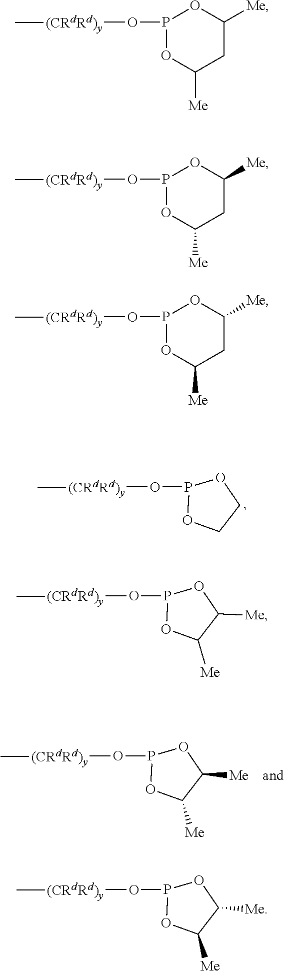

Many of these phosphate esters and phosphite esters are acid label and, when administered orally, metabolize to the corresponding phosphates and phosphites under the acidic conditions of the stomach and/or gut.

Thus, in the phosphorous-containing prodrugs described herein, the identity of the particular phosphorous-containing progroups R^{p} employed can be selected to tailor the prodrugs for particular modes of delivery, etc.

The suitability of any particular progroup R^{p} for a desired mode of administration can be confirmed in biochemical assays. For example, if a prodrug is to be administered by injection into a particular tissue or organ, and the identities of the various phosphatases expressed in the tissue or organ are known, the particular prodrug can be tested for metabolism in biochemical assays with the isolated phosphatase(s). Alternatively, the particular prodrug can be tested for metabolism to the active 2,4-pyrimidinediamine compound with tissue and/or organ extracts. Using tissue and/or organ extracts can be of particular convenience when the identity(ies) of the phosphatases expressed in the target tissues or organs are unknown, or in instances when the isolated phosphatases are not conveniently available. Skilled artisans will be able to readily select progroups R^{p} having metabolic properties (such as kinetics) suitable for particular applications using such in vitro tests. Of course, specific prodrugs could also be tested for suitable metabolism in in vitro animal models.

In some embodiments, the prodrugs are prodrugs according to structural formula (I) or (Ia) that have one or more features selected from:
(i) R⁵ is fluoro;
(ii) R² is a phenyl optionally substituted with one or more of the same or different R⁸ groups;
(iii) R² is 3,4,5-tri(loweralkoxy)phenyl;
(iv) R² is 3,4,5-trimethoxyphenyl;
(v) Y or Y¹ is O; Z¹ is CH, Z² is N; R¹⁷ and R¹⁸ are each methyl; and R¹⁹ and R²⁰ are taken together to form an oxogroup; and
(vi) R^{p} is a hydroxyalkyl-containing progroup of the formula —CH₂OH, or a phosphate-containing progroup of the formula —(CR^{d}R^{d})_{y}—O—P(O)(OH)₂, or a phosphate ester, phosphite or phosphite ester analog thereof, wherein y is 1 or 2 and each R^{d} is, independently of the others, selected from hydrogen and unsubstituted lower alkyl, or
(vii) R^{p} is selected from —CH₂OH, CH₂—SH, —CH₂—NH₂, —CH₂—NHR⁵⁰, —CH₂—N(R⁵⁰)₂, —CH₂-A-R^{t}, —CH₂-A-C(O)R^{f}, —CH₂-A-C(O)OR′ and —CH₂-A-C(O)NR^{t}R^{f}, where A, R⁵⁰ and R^{f} are as previously defined.

In some embodiments, the prodrugs of structural formulae (I) and (Ia) have two or three of the above-delineated features. In one specific embodiment, the prodrugs have features (i), (iii) and (v). In another specific embodiment, the prodrugs have features (i), (iv) and (v). In still another specific embodiment, the prodrugs have features (i), (iii), (v) and (vi) or (vii). In still another specific embodiment, the prodrugs have features (i), (iv), (v) and (vi) or (vii). In still another specific embodiment, R^{p} is a phosphate-containing progroup of the formula —(CR^{d}R^{d})_{y}—O—P(O)(OH)₂.

In all of the compounds described herein that include substituent alternatives that may be substituted, such as, for example, some of the substituent alternatives delineated for R^{d}, R^{e}, R^{f}, R⁹, R^{h}, Rⁱ and R^{j}, the substitutions are typically, independently of one another, selected from amongst the R^{b} groups described in connection with structural formula (I). In a specific embodiment, any present substitutions are, independently of one another, selected from hydroxyl, lower alkoxy, (C6-C14) aryloxy, lower alkoxyalkyl, methoxymethyl, methoxyethyl, ethoxymethyl, ethoxyethyl and halogen.

Those of skill in the art will appreciate that many of the prodrugs described herein, as well as the various prodrug species specifically described and/or illustrated herein, may exhibit the phenomena of tautomerism, conformational isomerism, geometric isomerism and/or optical isomerism. For example, the prodrugs may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the prodrugs may exist in several tautomeric forms, including the enol form, the keto form and mixtures thereof. As the various compound names, formulae and drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the prodrugs having one or more of the utilities described herein, as well as mixtures of these various different isomeric forms. In cases of limited rotation around the 2,4-pryimidinediamine moiety, atrop isomers are also possible and are also specifically included in the compounds of the invention.

Moreover, skilled artisans will appreciate that when lists of alternative substituents include members which, owing to valency requirements or other reasons, cannot be used to substitute a particular group, the list is intended to be read in context to include those members of the list that are suitable for substituting the particular group. For example, skilled artisans will appreciate that while all of the listed alternatives for R^{b} can be used to substitute an alkyl group, certain of the alternatives, such as ═O, cannot be used to substitute a phenyl group. It is to be understood that only possible combinations of substituent-group pairs are intended.

The prodrugs described herein may be identified by either their chemical structure or their chemical name. When the chemical structure and the chemical name conflict, the chemical structure is determinative of the identity of the specific prodrug.

Depending upon the nature of the various substituents, the prodrugs described herein may be in the form of salts. Such salts include salts suitable for pharmaceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, etc.).

The prodrugs described herein, as well as the salts thereof, may also be in the form of hydrates, solvates and N-oxides, as are well-known in the art. Unless specifically indicated otherwise, the expression “prodrug” is intended to encompass such salts, hydrates, solvates and/or N-oxides. Specific exemplary salts include, but are not limited to, mono- and di-sodium salts, mono- and di-potassium salts, mono- and di-lithium salts, mono- and di-alkylamino salts, mono-magnesium salts, mono-calcium salts and ammonium salts.

Additional aspects of prodrugs suitable for the invention are describe din U.S. Pat. No. 7,449,458, the disclosure of which is incorporated herein by reference.

### Pharmaceutical Compositions

In certain embodiments, the present disclosure provides pharmaceutical compositions that include a pharmaceutically acceptable carrier and a therapeutically effective amount of a compound or prodrug of the present disclosure or a pharmaceutically acceptable salt or solvate or stereoisomer thereof.

A pharmaceutical composition that includes a subject compound may be administered to a subject alone, or in combination with other supplementary active agents. For example, one or more compounds according to the present disclosure can be administered to a subject with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A subject compound or prodrug may be administered to a subject using any convenient means capable of resulting in the desired reduction in disease condition or symptom. Thus, a subject compound or prodrug can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, aerosols, and the like.

In certain embodiments, a subject compound or prodrug may be formulated as a pharmaceutical composition, where the pharmaceutical composition is an oral dosage formulation, such as a tablet. Additional aspects of oral dosage formulations (e.g., tablets) suitable for the invention are described in U.S. Pat. No. 8,771,648, the disclosure of which is incorporated herein by reference.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of disclosed compounds or prodrugs. Pharmaceutical compositions that include at least one of the subject compounds or prodrugs can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable carrier in addition to at least one active ingredient, such as a subject compound or prodrug. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. For example, parenteral formulations may include injectable fluids, such as, but not limited to, pharmaceutically and physiologically acceptable fluids such as water, physiological saline, balanced salt solutions, aqueous dextrose, glycerol or the like as a vehicle. For solid compositions (e.g., powder, pill, tablet, or capsule forms), non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other examples of excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) water (e.g., pyrogen-free water); (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound or prodrug. Examples of pharmaceutically acceptable salts include non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, methyl sulfonic acid, salicylic acid, formic acid, trichloroacetic acid, trifluoroacetic acid, gluconic acid, asparagic acid, aspartic acid, benzenesulfonic acid, para-toluenesulfonic acid, naphthalenesulfonic acid, combinations thereof, and the like. In certain embodiments, the pharmaceutically acceptable salt includes formic acid. Other examples of pharmaceutically acceptable salts include non-toxic salts of a free acid form of compounds or prodrugs according to the present disclosure. Such salts are derived from inorganic or organic bases. Pharmaceutically acceptable base addition salts include those derived from inorganic bases such as sodium, potassium, lithium, ammonium, calcium, magnesium, iron, zinc, copper, manganese, aluminum salts, combinations thereof, and the like. Examples of salts are the ammonium, potassium, sodium, calcium, and magnesium salts. Salts of the presently disclosed compounds or prodrugs can be derived from pharmaceutically acceptable organic non-toxic bases including, but not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, 2-amino-2-hydroxymethyl-propane-1,3-diol (“Tris” salt), dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, combinations thereof, and the like. Pharmaceutically acceptable salts are described further in S. M. Berge, et al., “Pharmaceutical Salts,” J. Pharm. Sci., 1977; 66:1-19 and Remington's Pharmaceutical Sciences, 19th Edition, Mack Publishing Company, Easton, Pa., 1995.

In certain embodiments, a subject compound or prodrug is formulated as a pharmaceutically acceptable salt, where the pharmaceutically acceptable salt is a disodium hexahydrate form of the subject compound or prodrug. Additional aspects of salts and hydrates of the subject compounds and prodrugs suitable for the invention are described in U.S. Pat. Nos. 8,163,902 and 8,445,485, the disclosures of which are incorporated herein by reference.

A subject compound or prodrug can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound or prodrug can be formulated into preparations for injection by dissolving, suspending or emulsifying the compound in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

A subject compound or prodrug can be utilized in aerosol formulation to be administered intrapulmonarily (e.g., via inhalation). A subject compound or prodrug can be formulated into pressurized acceptable propellants such as dichlorodifluoromethane, propane, nitrogen and the like.

Furthermore, a subject compound or prodrug can be made into suppositories by mixing with a variety of bases such as emulsifying bases or water-soluble bases. A subject compound or prodrug can be administered rectally via a suppository. The suppository can include vehicles such as cocoa butter, carbowaxes and polyethylene glycols, which melt at body temperature, yet are substantially solid at room temperature.

The term “unit dosage form,” as used herein, refers to physically discrete units suitable as unitary dosages for human and animal subjects, each unit containing a predetermined quantity of a subject compound or prodrug calculated in an amount sufficient to produce the desired effect in association with a pharmaceutically acceptable diluent, carrier or vehicle. The specifications for a subject compound or prodrug depend on the particular compound employed and the effect to be achieved, and the pharmacodynamics associated with each compound or prodrug in the host.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). In other embodiments, the subject compounds or prodrugs may be formulated for intrapulmonary administration. For example, intrapulmonary formulations of the subject prodrugs may include, but are not limited to, dry powder or solution formulations, and intrapulmonary formulations of the subject compounds may include, but are not limited to, dry powder or suspension formulations. Methods of preparing such dosage forms are known, or will be apparent, to those skilled in the art.

Certain embodiments of the pharmaceutical compositions that include a subject compound or prodrug may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compounds or prodrugs disclosed herein in an amount effective to achieve the desired effect in the subject being treated.

Each therapeutic compound or prodrug can independently be in any dosage form, such as those described herein, and can also be administered in various ways, as described herein. For example, the compounds or prodrugs may be formulated together, in a single dosage unit (that is, combined together in one form such as capsule, tablet, powder, or liquid, etc.) as a combination product. Alternatively, when not formulated together in a single dosage unit, an individual subject compound or prodrug may be administered at the same time as another therapeutic compound or prodrug or sequentially, in any order thereof.

A disclosed compound or prodrug can be administered alone, as the sole active pharmaceutical agent, or in combination with one or more additional compounds or prodrugs of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

### Combination Therapy

The present compound may be is administered in combination with one or more other therapeutic agents, the other therapeutic agents may target sickle cell disease. For example, the compound may be administered along with hydroxyurea (Droxia, Hydrea, Siklos), L-glutamine oral powder (Endari), crizanlizumab (Adakveo), voxelotor (Oxbryta) or another pain-relieving medication such as a narcotic.

### Clinical Treatment of Subjects Having or Suspected of Having SCD

This example describes an open label, dose-escalating, phase 1 clinical study conducted to assess the safety and tolerability of escalating doses of fostamatinib in subjects with sickle cell disease (SCD).

Subjects Selected for Fostamatinib Treatment are
aged between 18-65 years having a diagnosis of SCA (HbSS or HbSβ⁰) confirmed by hemoglobin electrophoresis performed on patients at least 90 days after a blood transfusion if previously transfused.
Patients having no transfusion in the 12 weeks prior, or absence of Hb A on hemoglobin analysis (by high-performance liquid chromatography; HPLC)
Have adequate organ function, as defined by:
   a. Serum aspartate aminotransferase (AST)≤1.5×Upper Limit of Normal (ULN) (unless the increased AST is assessed by the physician as due to hemolysis) and alanine aminotransferase (ALT)≤1.5×ULN.

$$Absolute neutrophil count \geq 1.5 \times 109 / L . \qquad b .$$

$$Hemoglobin \geq 7 g / dL . \qquad c$$

$$Platelet count \geq 100 \times 109 / L . \qquad d .$$

If on hydroxyurea, subject should be on stable dose of hydroxyurea (defined as a stable dose for at least 3 months and inclusive of dose modifications for hematological toxicity per discretion).

At the discretion of a physician skilled in the art, patients may be excluded that present the following:
a. History of neutropenia (benign ethnic neutropenia and/or acquired neutropenia unrelated to drug suppression by hydroxyurea and/or cyclic hematopoiesis).
b. History of posterior reversible encephalopathy syndrome (PRES)
c. History of poorly controlled hypertension (defined as systolic blood pressure ≥140 mmHg or average diastolic blood pressure ≥90 mmHg based on an average of 3 blood pressure readings despite adequate antihypertensive therapy) unless controlled for >90 days prior to enrollment.
d. Active viral infection as evidenced by testing positive for hepatitis B surface antigen or hepatitis C virus (HCV) antibody (ab) with signs of active hepatitis B or C virus infection. If the subject is positive for HCV Ab, a reverse transcriptase-polymerase chain reaction test will be conducted. Subjects with hepatitis C may be rescreened after receiving appropriate hepatitis C treatment.
e. History of drug-induced cholestatic hepatitis.
f. History of any primary malignancy.
g. Testing positive for human immunodeficiency virus 1 or 2 Ab with evidence for ongoing active infection (i.e., CD 4 count <400/μL and viral load >100,000 copies/ml) on antiretroviral therapy.
h. Use of newly approved SCD therapy (L-glutamine, voxelotor or crizanlizumab) is NOT recommended.
i. Having had a prior bone marrow or stem cell transplant.
j. Currently pregnant or lactating.
k. Currently receiving strong inhibitors of CYP3A4/5 that have not been stopped for ≥5 days or a time frame equivalent to 5 half-lives (whichever is longer), or strong inducers of CYP3A4 that have not been stopped for ≥28 days or a time frame equivalent to 5 half-lives (whichever is longer), prior to signing consent. SCD patients that are receiving treatment with CYP3A4 substrate drugs, some BCRP substrate drugs (eg. rosuvastatin), and some P-glycoprotein substrate drugs (eg. Digoxin) are not recommended.
l. Currently receiving erythropoiesis stimulating agents.

### Treatment Regimen

Outpatients with SCD will receive fostamatinib at a dose of 100 mg BID for 14 days which will be escalated, depending on tolerability, to a dose of 150 mg BID for 28 days.

### Dosing and Administration

Patients initially receive fostamatinib at a dose of 100 mg orally BID for a total of 14 days to be swallowed as an oral tablet. Dose escalation and modifications based on adverse reactions may be considered as known to those of skill in the art, considering Tables 1, 2 and 3, below.

Those patients tolerating 100 mg BID doses without experiencing new onset adverse events related to hypertension, hepatotoxicity, diarrhea, and neutropenia may have their fostamatinib dose escalated to 150 mg BID. Those patients experiencing grade 1-3 adverse events related to hypertension, hepatotoxicity and diarrhea that return to baseline after withholding and remain normal after resuming fostamatinib, may have their fostamatinib dose escalated to the 150 mg BID level. Those patients experiencing grade 2 or 3 adverse events related to hypertension, hepatotoxicity, neutropenia, and diarrhea that fail to return to baseline or ≤grade 1 after withholding fostamatinib for >7 days may not be escalated to the 150 mg BID level. Toxicity experienced at each individual dose level and the ability to successfully escalate to a therapeutically effective dose, such as the 150 mg BID dose will be assessed.

TABLE 1
Dose Escalation

| Adverse Reaction | Recommended action |
|---|---|
| Hypertension | |
| Stage 1: systolic between 130-139 or | Initiate or increase antihypertensive |
| diastolic between 80-89 mmHg | medication for patients with increased risk of |
| | cardiovascular risk and adjust as needed. No |
| | dose modification to fostamatinib required. |
| Stage 2: systolic at least 140 or diastolic at | Increase antihypertensive medication and |
| least 90 mmHg | adjust as needed until BP control. No dose |
| | modification to fostamatinib required. |
| Hypertension with systolic >140 or diastolic | Increase current antihypertensive medication |
| >90 mmHg requiring addition of new | and add new medication adjusting as needed |
| antihypertensive medication. | until BP control to mitigate drug toxicity. |
| | Permanently discontinue fostamatinib. |
| Hypertensive crisis: systolic over 180 and | Increase antihypertensive medication and |
| or diastolic over 120 mmHg | adjust as needed until BP control to mitigate |
| | drug toxicity. Permanently discontinue |
| | fostamatinib. |
| Hepatotoxicity | |
| Increase in AST/ALT by x2.5 from level at | Withhold fostamatinib and recheck liver |
| the time of enrollment but <3x upper limit | function tests the next day. Resume |
| of normal (ULN). | fostamatinib when AST/ALT have returned to |
| | bascline or ≤Grade 1. |
| Increase in AST/ALT to > 3x upper limit of | Withhold fostamatinib and recheck liver |
| normal to ≤5x ULN and total BL (bilirubin) | function tests the next day. Resume |
| greater than 2x upper limit of normal (ULN) | fostamatinib when AST/ALT and bilirubin |
| | returns to baseline or ≤ Grade 1. Resume |
| | fostamatinib at lower dosage or if toxicity |
| | occurred at 100 mg dosage, permanently |
| | discontinue fostamatinib. |
| Elevated unconjugated (indirect) BL in | Continue fostamatinib with frequent |
| absence of other LFT abnormalities | monitoring since isolated increase in |
| | unconjugated (indirect) bilirubin may be due |
| | to UGT1A1 inhibition |
| Increase in AST/ALT > 5x ULN | Permanently discontinue fostamatinib. |
| Diarrhea | |
| Diarrhea Grade 1 and 2 | Manage diarrhea using supportive measures |
| | (e.g., dietary changes, hydration and/or |
| | antidiarrheal medication) early after the onset |
| | until symptoms resolve. |
| Diarrhea Grade 3 | For diarrhea at least possibly related to |
| | fostamatinib, withhold fostamatinib and |
| | resume at lower dosage once symptoms have |
| | improved to baseline or ≤ Grade 1. If diarrhea |
| | occurred at the 100 mg dose, permanently |
| | discontinuc fostamatinib. |
| Diarrhea Grade 4 | For diarrhea considered at least possibly |
| | related to fostamatinib, permanently |
| | discontinue fostamatinib. |
| Neutropenia | |
| Neutropenia | If absolute neutrophil count (ANC) decreased |
| | to less than 1 × 10\hat{ }9/L, withhold fostamatinib. |
| | Resume fostamatinib when ANC ≥ 1.5 × |
| | 10\hat{ }9/L at a lower dosage or if toxicity |
| | occurred at 100 mg dosage, permanently |
| | discontinue fostamatinib. |
| Other AEs** | |
| Grade 3 AEs possibly related to fostamatinib | Withhold fostamatinib until return to baseline |
| | or ≤Grade 1. |
| | If toxicity occurred at 100 mg BID dose, do |
| | not escalate fostamatinib to level 2. |
| For recurrent grade ≥3 events or events that | Permanently discontinue fostamatinib. |
| do not return to baseline or ≤Grade 1 within | |
| 4 weeks | |
| Grade 4 AEs possibly related to fostamatinib | Permanently discontinue fostamatinib |

*Fostamatinib dose may be decreased anytime at the discretion of physician in relation to any adverse or perceived adverse event irrespective of the attribution to the fostamatinib.
**Those patients experiencing a VOC or has new onset clinical symptoms deemed related totheir underlying sickle cell disease will have fostamatinib discontinued per physician discretion.

### Anemia

Anemia is an extremely common complication of SCD. While we expect anemia to remain unchanged with fostamatinib treatment, there are rare situations in which a participant's anemia may worsen to below baseline. For these events, attribution to research and need for dose adjustments of fostamatinib will largely depend on the etiology. Table 2 summarizes recommended dose modification associated with anemia.

TABLE 2
Anemia and Dose Modification

| Anemia Category | Attribution | Dose Modification |
|---|---|---|
| Anemia in | N/A (captured in the | None required |
| the setting | overarching AE for | |
| of a VOC | VOC and not | |
| | reported separately) | |
| Unexplained | Dependent on | None required |
| worsening of | whether another | Perform workup |
| baseline anemia | etiology is identified | for anemia |
| Any Grade 4 | Dependent on | Consider discontinuing |
| anemia | whether another | fostamatinib, |
| | etiology | dependent on |
| | is identified | etiology of anemia. |

### Neutropenia

Neutropenia is not a complication of SCD but can occur with hydroxyurea treatment, especially at maximally tolerated dose. We expect that hydroxyurea related neutropenia may be worsened with fostamatinib treatment. For these events, attribution to research and need for dose adjustments of fostamatinib will largely depend on the etiology. Table 3 summarizes recommended dose modification associated with neutropenia.

TABLE 3
Neutropenia and Dose Modification

| Neutropenia | | |
|---|---|---|
| Category | Attribution | Dose Modification |
| Neutropenia while on | N/A (captured in the | Dose reduction or |
| stable hydroxyurea | overarching AE for | discontinuation |
| treatment in the | VOC and not | depending |
| setting of a VOC | reported separately) | on severity |
| Unexplained | Dependent on | Dose reduction |
| worsening of | whether another | or discontinuation |
| | | depending on severity |
| baseline neutropenia | etiology is | Perform workup for |
| due to hydroxyurea | identified | neutropenia |
| Any Grade 4 | Dependent on | Consider discontinuing |
| neutropenia | whether another | fostamatinib, |
| irrespective of | etiology is | dependent on |
| hydroxyurea | identified | etiology of anemia |
| treatment | | |

### Drug Administration

Fostamatinib must be taken whole and may be taken with or without food. The fostamatinib should be taken at a fixed time (e.g., 9:00 AM and 9:00 PM according to subject convenience). If a dose is missed and more than 1 hour has passed, then the patient should skip the dose and wait until the next dose's regularly scheduled time.

### Biomarker Evaluation

Various biomarkers and changes in the same may inform treatment of SCD with fostamatinib. These biomarkers are relevant to the mechanism of action of fostamatinib on thromboinflammation pathophysiology and red cell metabolism and are relevant to drug efficacy, dose modification, and selection of patients for continued treatment. In particular, assessment of the following criteria inform the treatment regimen:
Neutrophil activation and NETosis at regular time intervals on fostamatinib and change from baseline.
Platelet activation and function at regular time intervals on fostamatinib and change from baseline.
Percentage of sickled cells and time to 50% sickling (t50) under normal and hypoxic ex vivo conditions at regular time intervals on fostamatinib and change from baseline.
Evaluate effect of fostamatinib on RBC band 3 tyrosine phosphorylation, RBC metabolism.
Change from baseline in intracellular reactive oxidative species (ROS) in RBCs at different doses of fostamatinib.
Markers of coagulation activation at regular time intervals on fostamatinib and change from baseline.

Table 4 includes recommended biospecimen evaluations and timing.

TABLE 4
Biomarker Evalation

| | | | | Freqency** |
|---|---|---|---|---|
| | | | | (D day relative |
| | | | | to first |
| | Volume | | | treatment |
| | blood | | | with |
| Test/assay | (approx.) | | Type of tube | fostamatinib) |
| CBC (with | 3 | mL | EDTA tube | D0, 14, 28, |
| differential) | | | (lavender) | 42 and 70 |
| Reticulocyte | 4 | mL | Lithium Heparin | |
| count | | | tube | |
| BMP | | | (light green) | |
| LFT | | | | |
| PT | 3 | mL × 2 | Sod Citrate | D0, 14, 28, |
| PTT | | | (BLU) x2 | 42 and 70 |
| INR | | | | |
| Fibrinogen | | | | |
| D-dimer | | | | |
| Von Willebrand | | | | |
| factor | | | | |
| VIII level | | | | |
| P-selectin | 3 | mL | Sod Citrate | D0, 14, 28, |
| TAT | | | (BLU) | 42 and 70 |
| Plasma Tissue | | | | |
| Factor | | | | |
| PDI | | | | |
| Neutrophils | 3 | mL × 2 | Sod Citrate | D0, 14, 28, |
| | | | (BLU) | 42 and 70 |
| PBMC & | 5 | mL | Sodium Heparin | D0, 14, 28, |
| neutrophil | Use | | Green top | 42 and 70 |
| signaling | sample | | | |
| Aggregates | above | | | |
| RBC metabolism | 3 | mL × 1 | EDTA | D0, 14, 28, |
| | | | (lavender) | 42 and 70 |
| RBC oxygen | 3 | mL | EDTA | D0, 14, 28, |
| binding | | | (lavender) | 42 and 70 |

**Day 0 labs should be drawn prior to first fostamatinib administration.
Special tubes will be provided by the research laboratory.

### Additional Clinical Assessment

The clinical efficacy and safety of different doses of fostamatinib in subjects with stable sickle cell disease can be assessed in part by examining the frequency and severity of AEs, and changes in laboratory parameters, including levels in hemoglobin, reticulocyte counts, bilirubin and lactate dehydrogenase, white blood cell count, absolute neutrophil count, and platelet count.

Factors for consideration:
a. the type, incidence, severity, and relationship to fostamatinib treatment of AEs and serious adverse events (SAEs).
b. number of discontinuations due to AEs.
c. results of clinical laboratory tests over time (e.g., serum chemistry, liver function test, hematology, coagulation).
d. change from baseline in hemoglobin; reticulocyte count; LDH; serum bilirubin; white blood cell count; absolute neutrophil count; platelet count; physical examination findings; and vital signs.
e. Proportion of patients receiving hydroxyurea that experience a >50% reduction in absolute neutrophil count.

The mechanistic actions of fostamatinib on cellular activity and pathophysiology can be assessed by comparisons of baseline activities to those observed after fostamatinib dosing, for example, dosing at 100, 150 or 200 mg.

Points for consideration are:
a. Change from baseline in oxygen binding p50 value at different doses of fostamatinib.
b. Percentage of sickled cells under normal and hypoxic ex vivo conditions at different doses of fostamatinib.
c. Change from baseline in neutrophil activation markers and NETosis at different doses of fostamatinib.
d. Change from baseline in platelet activation markers and platelet function tests at different doses of fostamatinib.
e. Change from baseline in red cell metabolism studies at different doses of fostamatinib.
f. Change from baseline in markers of coagulation at different doses of fostamatinib.

As outlined herein, intracellular signaling pathways that are affected in neutrophils and RBCs after fostamatinib administration can be assessed, for example, in biochemical studies interrogating neutrophil and RBC signaling pathways relevant to inhibition of SYK phosphorylation and Band 3 tyrosine kinase phosphorylation at different doses of fostamatinib.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treatment, comprising:
administering to subject having or suspected of having sickle cell disease, a compound, wherein the compound is fostamatinib of the formula: 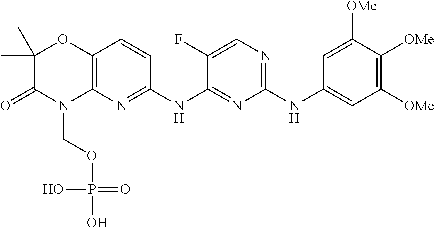 [SMILES: COc1cc(Nc2ncc(F)c(Nc3ccc4c(n3)N(COP(=O)(O)O)C(=O)C(C)(C)O4)n2)cc(OC)c1OC]
or a pharmaceutically acceptable salt thereof, or 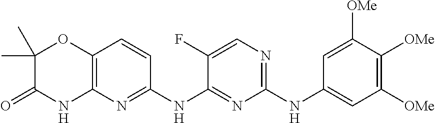 [SMILES: COc1cc(Nc2ncc(F)c(Nc3ccc4c(n3)NC(=O)C(C)(C)O4)n2)cc(OC)c1OC]
or a pharmaceutically acceptable salt thereof.

2. The method of claim 1, wherein the subject is exhibiting one or more of the following symptoms: anemia, sickle cell crisis, vaso-occlusive crisis, splenic sequestration crisis, splenic sequestration crises, acute chest syndrome, acute chest syndrome, aplastic crisis, haemolytic crisis, dactylitis, pneumonia, respiratory infection, bone-marrow embolisation, or atelectasis.

3. The method of claim 1, wherein the administering is systemically administering.

4. The method of claim 3, wherein the administering is done orally or intravenously.

5. The method of claim 1, wherein the administering is done by pulmonary administration.

6. The method of claim 5, wherein the administering is done using an inhaler or nebulizer.

7. The method of claim 1, wherein the subject is in intensive care.

8. The method of claim 1, wherein the active component is of the formula:

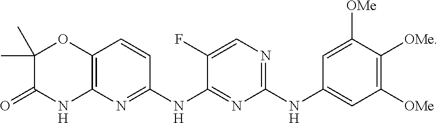 [SMILES: COc1cc(Nc2ncc(F)c(Nc3ccc4c(n3)NC(=O)C(C)(C)O4)n2)cc(OC)c1OC]

9. The method of claim 1, further comprising measuring neutrophil activation.

10. The method of claim 1, further comprising measuring platelet count.

11. The method of claim 1, further comprising measuring the percentage of sickled cells.

12. The method of claim 1, further comprising measuring sickling kinetics.

13. The method of claim 1, wherein neutrophil activation is reduced following administration of fostamatinib.

14. The method of claim 1, wherein administering fostamatinib results in increased platelet count.

15. The method of claim 1, further comprising measuring reactive oxygen species (ROS).

16. The method of claim 1, wherein administering fostamatinib results in reduced (ROS).

17. A method of treatment, comprising:
administering to a subject having or suspected of having sickle cell disease a compound; and
taking a blood sample from the subject, wherein the compound is fostamatinib of the formula: 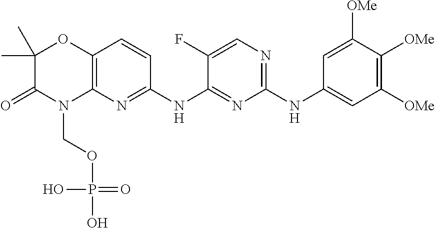 [SMILES: COc1cc(Nc2ncc(F)c(Nc3ccc4c(n3)N(COP(=O)(O)O)C(=O)C(C)(C)O4)n2)cc(OC)c1OC]
or a pharmaceutically acceptable salt thereof, or 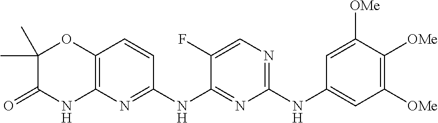 [SMILES: COc1cc(Nc2ncc(F)c(Nc3ccc4c(n3)NC(=O)C(C)(C)O4)n2)cc(OC)c1OC]
or a pharmaceutically acceptable salt thereof.

18. The method of claim 17, further comprising measuring neutrophil count, neutrophil activation, or both in the blood sample.

19. The method of claim 18, further comprising measuring platelet count in the blood sample.

20. The method of claim 17, further comprising measuring (ROS).

21. The method of claim 17, further comprising administering hydroxyurea to the subject.

22. The method of claim 17, wherein the subject exhibits neutropenia; and the fostamatinib dosage is reduced.

23. The method of claim 22, wherein the fostamatinib dosage is reduced from 200 mg BID.

24. The method of claim 22, wherein the fostamatinib dosage is reduced to 150 mg BID.

25. The method of claim 22, wherein the fostamatinib dosage is reduced from 150 mg BID.

26. The method of claim 22, wherein the fostamatinib dosage is reduced to 100 mg BID.

27. The method claim 22, wherein the fostamatinib dosage is maintained at 100 mg BID.

28. The method of claim 22, wherein the dosage is reduced to 150 mg QD.

29. The method of claim 17, wherein fostamatinib is administered in an amount of 150 mg QD.

30. The method of claim 17, wherein fostamatinib is administered in an amount of 100 mg QD.
